(19) United States
(12) Patent Application Publication
(10) Pub. No.: US 2010/0143805 A1
(43) Pub. Date: Jun. 10, 2010

(54) NITROXIDES FOR LITHIUM-ION BATTERIES

(75) Inventors: Tobias Hintermann, Therwill (CH); Peter Nesvadba, Marly (CH); Markus Frey, Rheinfelden (CH); Lucienne Bugnon Folger, Pfeffingen (CH)

Correspondence Address: BASF Performance Products LLC;Patent Department, 540 White Plains Road, P.O. Box 2005, Tarrytown, NY, 10591, US

(73) Assignee: CIBA CORPORATION, TARRYTOWN (NY)

(21) Appl. No.: 12/529,720

(22) PCT Filed: Feb. 29, 2008
(86) PCT No.: PCT/EP08/52464

§ 371 Date: Sep. 3, 2009

(30) Foreign Application Priority Data
Mar. 9, 2007 (EP) 07103869.9
Oct. 26, 2007 (EP) 07119346.0

(51) Int. Cl. H01M 6/16; H01M 10/05; H01M 10/26; C07D 241/04; C07F 9/06; C07D 233/54

(52) U.S. Cl. 429/338; 429/336; 429/337; 429/339; 429/341; 429/342; 429/203; 429/207; 544/358; 548/112; 548/335.1; 548/341.1

(57) ABSTRACT

This invention relates to overcharge protection and molecular redox shuttles in rechargeable lithium-ion cells. For this, specific nitroxyls or oxoammonium salts are used in the electrolyte. This invention also relates to a method of producing such lithium-ion cells and to a method of recharging such lithium-ion cells. This invention also pertains to some nitroxyls compounds and oxoammonium salts.

This invention relates to overcharge protection and molecular redox shuttles in rechargeable lithium-ion cells. For this, specific nitroxyls or oxoammonium salts are used in the electrolyte. This invention also relates to a method of producing such lithium-ion cells and to a method of recharging such lithium-ion cells. This invention also pertains to some nitroxyls compounds and oxoammonium salts.

WO-A-2006/124389 describes cycloaliphatic N-oxides as redox shuttles (i.e. protection against overcharge) for rechargeable lithium-ion cells. The cycloaliphatic N-oxide comprises a piperidinyl or a pyrrolidinyl ring.

EP-A-1843426 and WO2007/116363 describe among others cycloaliphatic N-oxides as redox active compounds dissolved in the electrolyte of rechargeable lithium-ion cells.

Q. Wang et al., Angew. Chem. Int. Ed. 2006, 45, 8197-8200 describes molecular redox shuttles for rechargeable lithium-ion cells. Osmium complexes are used as such molecular redox shuttles.

JP-A-2002-268861 describes secondary batteries with a 2,2,6,6-tetrasubstituted-piperidine-N-oxide or a 2,2,5,5-tetrasubstituted-pyrrolidine-N-oxide containing non-aqueous electrolyte.

EP-A-1381100 describes a charge storage device with a positive electrode comprising a 2,2,6,6-tetrasubstituted-piperidine-N-oxoammonium cation, a 2,2,5,5-tetrasubstituted-pyrrolidine-N-oxoammonium cation or a 2,2,5,5-tetrasubstituted-3-pyrroline-N-oxoammonium cation.

U.S. Pat. No. 3,532,703 describes 2,2,5,5-tetrasubstituted-4-oxoimidazolidine-1-oxides as stabilizers for polyolefins against deterioration resulting from exposure to light.

WO-A-01/23435 describes 2-oxo-3,3,5,5-tetrasubstituted-morpholine-N-oxides as polymerization regulator.

When properly designed and constructed, rechargeable lithium-ion cells can exhibit excellent charge-discharge cycle life, little or no memory effect, and high specific and volumetric energy. However, lithium-ion cells do have some shortcomings, including an inability to tolerate recharging to potentials above the manufacturer's recommended end of charge potential without degradation in cycle life; the danger of overheating, fire or explosion for cells recharged to potentials above the recommended end of charge potential; and difficulties in making large cells having sufficient tolerance to electrical and mechanical abuse for consumer applications. Single and connected (for example, series-connected) lithium-ion cells typically incorporate charge control electronics to prevent individual cells from exceeding the recommended end of charge potential. This circuitry adds cost and complexity and has discouraged the use of lithium ion cells and batteries in low-cost mass market electrical and electronic devices such as flashlights, radios, CD players and the like. Instead, these low-cost devices typically are powered by non-rechargeable batteries such as alkaline cells.

Various chemical compounds have been proposed for imparting overcharge protection to rechargeable lithium-ion cells. Chemical compounds designated as “redox shuttles” or “shuttles” may in theory provide an oxidizable and reducible charge-transporting species that may repeatedly transport charge between the negative and positive electrodes once the charging potential reaches a desired value.

The electroactive materials in lithium-ion batteries must be electrochemically addressable for their capacity to be explored fully. Owing to a lack of electronic conductivity of the electrode material, a large amount of conducting additive, for example carbon black or graphite, has to be incorporated into the electrode to form a continuous conducting network for electron percolation. Consequently, the energy density of the battery is greatly decreased by the presence of a large volume of inactive conducting agent. Molecular redox targeting by freely diffusing relay molecules can help to overcome the problem of insulating or poorly conducting lithium-insertion materials.

The phrase “positive electrode” refers to one of a pair of rechargeable lithium-ion cell electrodes that under normal circumstances and when the cell is fully charged will have the highest potential. We retain this terminology to refer to the same physical electrode under all cell operating conditions even if such electrode temporarily (e.g, due to cell overdischarge) is driven to or exhibits a potential below that of the other (the negative) electrode.

The phrase “negative electrode” refers to one of a pair of rechargeable lithium-ion cell electrodes that under normal circumstances and when the cell is fully charged will have the lowest potential. We retain this terminology to refer to the same physical electrode under all cell operating conditions even if such electrode is temporarily (e.g, due to cell overdischarge) driven to or exhibits a potential above that of the other (the positive) electrode.

The phrase “redox chemical shuttle” refers to an electrochemically reversible species that during charging of a lithium-ion cell can become oxidized at the positive electrode, migrate to the negative electrode, become reduced at the negative electrode to reform the unoxidized (or less-oxidized) shuttle species, and migrate back to the positive electrode.

The term “molecular redox shuttle for redox targeting” or “molecular redox shuttle” refers to an electrochemically reversible species. During charging, the molecular redox shuttle (S) for redox targeting is oxidized at the current collector. The oxidized species (S+), delivers the positive charge to the corresponding particles of the active electrode material, for example LiFePO₄, by bulk diffusion and are reduced back to S. By contrast, during the discharging process, S+ is reduced at the current collector to S, which in turn delivers electrons to the oxidized active electrode material. The advantage of using a freely diffusing redox shuttle is that it allows charge transport to proceed at a much faster rate, thus enhancing greatly the power output of the battery. So for instance, the response time of the electrodes can be reduced. For example, the amount of conducting additive (e.g. carbon black or graphite) in the electrodes can be reduced or omitted.

When used with respect to a positive electrode, the phrase “recharged potential” refers to a value E_{cp} measured relative to Li/Li⁺ by constructing a cell containing the positive electrode, a lithium metal negative electrode and an electrolyte but no compound (iii), carrying out a charge/discharge cycling test and observing the potential at which the positive electrode becomes delithiated during the first charge cycle to a lithium level corresponding to at least 90% of the available recharged cell capacity. For some positive electrodes (for example, LiFePO₄), this lithium level may correspond to approximately complete delithiation (for example, to Li₀FePO₄). For other positive electrodes (for example, some electrodes having a layered lithium-containing structure), this lithium level may correspond to partial delithiation.

The word “cyclable” when used in connection with a redox chemical shuttle refers to a material that when exposed to a charging voltage sufficient to oxidize the material (for example, from a neutral to a cationic form, or from a less-oxidized state to a more oxidized state) and at an overcharge charge flow equivalent to 100% of the cell capacity will provide at least two cycles of overcharge protection for a cell containing the chosen positive electrode.

The term “phase” refers to a homogeneous liquid portion that is present or that can form in a liquid system. The term “phases” refers to the presence of more than one phase in a heterogeneous liquid system. When used with respect to a mixture of a redox chemical shuttle and electrolyte, the terms “dissolved” and “dissolvable” refer to a shuttle that when present in or added to the electrolyte forms or will form a single phase solution containing a mobile charge-carrying species in an amount sufficient to provide overcharge protection at a charging current rate sufficient to charge fully in 10 hours or less a lithium-ion cell containing the chosen positive electrode, negative electrode and electrolyte.

When used with respect to a redox chemical shuttle, the phrase “oxidation potential” refers to a value E_{cv}. E_{cv} may be measured by dissolving the shuttle in the chosen electrolyte, measuring current flow vs. voltage using cyclic voltammetry and a platinum or glassy carbon working electrode, a copper counter electrode and a nonaqueous Ag/AgCl reference electrode and determining the potentials Vᵤₚ (i.e. during a scan to more positive potentials) and V_{down} (i.e. during a scan to more negative potentials), at which peak current flow is observed. E_{cv} will be the average of Vᵤₚ and V_{down}. Shuttle oxidation potentials may be closely estimated (to provide a value E_{calc}) by constructing a cell containing the shuttle, carrying out a charge/discharge cycling test, and observing during a charging sequence the potential at which a voltage plateau indicative of shuttle oxidation and reduction occurs. Shuttle oxidation potentials may be approximated (to provide a value “Ecajc”) using modeling software such as GAUSSIAN 03™ from Gaussian Inc. to predict oxidation potentials (for example, for compounds whose E_{cv} is not known) by correlating model ionization potentials to the oxidation potentials and lithium-ion cell behavior of measured compounds.

### DESCRIPTION OF THE FIGURES

FIG. 1: Reversible cyclovoltammograms of Cmpd 1

FIG. 2: Reversible cyclovoltammograms of Cmpd 9

FIG. 3: Reversible cyclovoltammograms of Cmpd 16

FIG. 4: Reversible cyclovoltammograms of Cmpd 20

FIG. 5: Reversible cyclovoltammograms of Cmpd 25

FIG. 6: Reversible cyclovoltammograms of Cmpd 26

FIG. 7: Reversible cyclovoltammograms of Cmpd 39

FIG. 8: Plot showing cell potential during successive charge-discharge cycles of the cell described in example with Cmpd 31.

The invention provides in one aspect a rechargeable lithium-ion cell comprising:

(a) a positive electrode (e.g. having a recharged potential),
(b) a negative electrode and
(c) an electrolyte comprising
(i) a lithium salt,
(ii) a polar aprotic solvent and
(iii) at least one compound selected from the group consisting of formula (d1)-(d6)

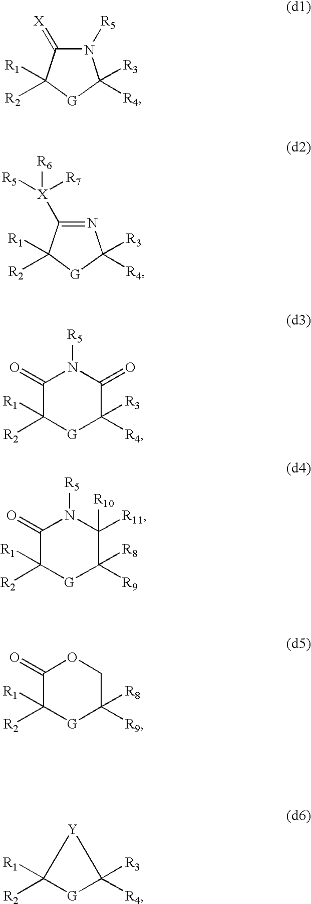

wherein

### G is

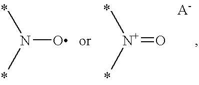

preferably G is

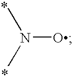

### X is O or S;

### if X is O,

then R₆ and R₇ are electron pairs;

### if X is S,

then R₆ and R₇ are independently an electron pair or ═O;
Y is —CH₂—O—CH₂—, —CH₂—S—CH₂—, —CH₂—S(═O)—CH₂—, —CH₂—S(═O)₂—CH₂—,

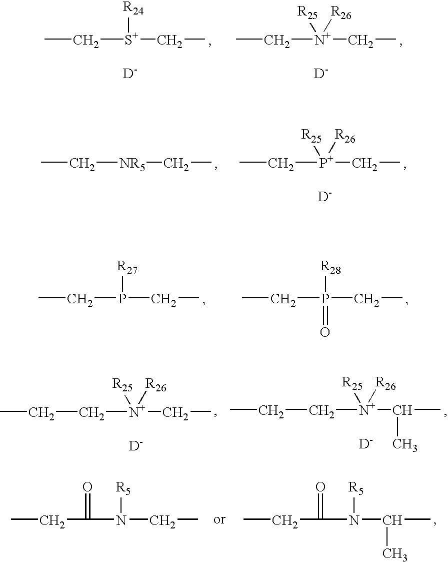

preferably —CH₂—S(═O)₂—CH₂—;
A⁻ and D⁻ are independently an anion of an organic or inorganic acid, preferably the anion of LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆H₅)₄, LiB(C₆F₅)₄, LiSbF₆, LiAsF₆, LiBr, LiBF₃C₂F₅ or LiPF₃(CF₂CF₃)₃, for instance D⁻ is I⁻, for example D⁻ is ClO₄⁻,
* indicates a free valence;
R₁, R₂, R₃ and R₄ are independently C₁-C₁₈alkyl, C₆-C₁₀aryl, C₅-C₈heteroaryl, C₇-C₁₁aralkyl or C₅-C₆-cycloalkyl; or said groups substituted by one or more F; or the said alkyl and/or cycloalkyl interrupted by one or more heteroatomgroup, preferably by O, NR₁₆, Si(R₁₆)(R₁₇), PR₁₆ or S, most preferably by O or NR₁₆; or the said alkyl and/or cycloalkyl substituted by one or more heteroatomgroup, preferably by Cl, —COOR₁₂, —CONHR₁₆, —CON(R₁₆)(R₁₇), OR₁₂, —OC(O)R₁₂, —OC(O)OR₁₂, —OC(O)NHR₁₆, —OC(O)N(R₁₆)(R₁₇), —NHC(O)R₁₆, —NR₁₆C(O)R₁₇, —NCO, —N₃, NHC(O)NHR₁₆, —NR₁₈C(O)N(R₁₆)(R₁₇), —NHCOOR₁₂, —N(R₁₆)(R₁₇), —NR₁₆COOR₁₂, —N⁺(R₁₆)(R₁₇)(R₁₈) A⁻, S⁺(R₁₆)(R₁₇) A⁻ or P⁺(R₁₆)(R₁₇)(R₁₈) A⁻; or the said alkyl and/or cycloalkyl both interrupted by and substituted by one or more heteroatomgroups (e.g. the ones defined above); or said aryl, heteroaryl and/or aralkyl substituted by 1 to 4 C₁-C₄alkyl; or
R₁ and R₂ and/or R₃ and R₄ form together with the linking carbon atom a C₄-C₁₃cycloalkylbiradical which is unsubstituted or substituted by F; for instance, R₁-R₄ are CH₃:
R₅ is H, OH, C₁-C₁₈ alkyl, C₆-C₁₀aryl, C₇-C₁₁aralkyl, C₂-C₁₈alkenyl, C₂-C₁₈alkinyl, C₅-C₆cycloalkyl, glycidyl, —CO—R₁₆, —CO—NH—R₁₆, —CON(R₁₆)(R₁₇), —O—CO—R₁₆, CO—OR₁₂, —PO(OR₁₂)(OR₁₃), —S(═O)₂OR₁₂, —SR₁₂, —S(═O)R₁₂, —S(═O)₂R₁₂, —S—OR₁₂, —S(═O)—OR₁₂, —SiR₁₆R₁₇R₁₈, —CN or halogen; or said groups substituted by one or more F; or said alkyl, alkenyl, alkinyl or cycloalkyl interrupted by one or more heteroatomgroup, preferably by O, NR₁₆, Si(R₁₆)(R₁₇), PR₁₆ or S, most preferably by O or NR₁₆; or the said alkyl, alkenyl, alkinyl or cycloalkyl substituted by one or more heteroatomgroup, preferably by Cl, —COOR₁₂, —CONHR₁₆, —CON(R₁₆)(R₁₇), OR₁₂, —OC(O)R₁₆, —OC(O)OR₁₂, —OC(O)NHR₁₆, —OC(O)N(R₁₆)(R₁₇), —NHC(O)R₁₆, —NR₁₆C(O)R₁₇, —NCO, —N₃, NHC(O)NHR₁₆, —NR₁₈C(O)N(R₁₆)(R₁₇), —NHCOOR₁₂, —N(R₁₆)(R₁₇), —NR₁₆COOR₁₂, —N⁺(R₁₆)(R₁₇)(R₁₈) A⁻, S⁺(R₁₆)(R₁₇) A⁻ or P⁺(R₁₆)(R₁₇)(R₁₈) A⁻, more preferably by —O—CO—R₁₆, CO—OR₁₆ or OR₁₂, most preferably by OH; or the said alkyl and/or cycloalkyl both interrupted by and substituted by one or more heteroatomgroups (e.g. the ones defined above); or said aryl or aralkyl substituted by 1 to 4 C₁-C₄alkyl; or R₅ is a multivalent core with more than one structural units (d1)-(d4) or (d6) attached, the multivalent core is preferably a C₂-C₂₀ polyacyl from di-, tri-, tetra-, penta- or hexa-carboxylic acid), C₂-C₂₀ alkyl, C₆-C₁₀aryl, C₃-C₈heteroaryl, C₄-C₂₄ bi-, tri-, or tetra-aryl or C₄-C₂₄ bi-, tri- or tetra-heteroaryl, whereby the said groups are unsubstituted or substituted by F and/or the said polyacyl or said alkyl is uninterrupted or interrupted by one or more heteroatomgroup, preferably by O, NR₁₆, Si(R₁₆)(R₁₇), PR₁₆ or S, most preferably by O or NR₁₆, and/or the said polyacyl or said alkyl is unsubstituted or substituted by one or more heteroatomgroup, preferably by Cl, —COOR₁₂, —CONHR₁₆, —CON(R₁₆)(R₁₇), OR₁₂, —OC(O)R₁₆, —OC(O)OR₁₂, —OC(O)NHR₁₆, —OC(O)N(R₁₆)(R₁₇), —NHC(O)R₁₆, —NR₁₆C(O)R₁₇, —NCO, —N₃, NHC(O)NHR₁₆, —NR₁₈C(O)N(R₁₆)(R₁₇), —NHCOOR₁₂, —N(R₁₆)(R₁₇), —NR₁₆COOR₁₂, —N⁺(R₁₆)(R₁₇)(R₁₈) A⁻, S⁺(R₁₆)(R₁₇) A⁻ or P⁺(R₁₆)(R₁₇)(R₁₈) A⁻, most preferably by OH;
R₈ and R₉ are independently —CH₂O—CO—C₁-C₁₈alkyl, —CH₂—NH—CO—C₁-C₁₈alkyl or as defined for R₁;
or R₈ and R₉ form together with the linking carbon atom a

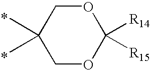

group;
R₁₀ and R₁₁ are independently H or CH₃;
R₁₂ and R₁₃ are independently H, NH₄, Li, Na, K or as defined for R₁₆;
R₁₄, R₁₅ are independently H or C₁-C₁₈ Alkyl;
or R₁₄ and R₁₅ form together with the linking carbon atom a C₄-C₁₃cycloalkylbiradical;
R₁₆, R₁₇ and R₁₈ are independently C₁-C₁₈alkyl, C₂-C₁₈alkenyl, C₆-C₁₀aryl, C₅-C₈heteroaryl, C₇-C₁₁aralkyl or C₅-C₆cycloalkyl; or said groups substituted by one or more F; or the said alkyl and/or cycloalkyl interrupted by one or more heteroatomgroup, preferably by O, NR₂₀, Si(R₂₀)(R₂₁), PR₂₀ or S, most preferably by O or NR₂₀; or said alkyl and/or cycloalkyl is substituted by one or more heteroatomgroup, preferably by Cl, —COOR₂₃, —CONHR₂₀, —CON(R₂₀)(R₂₁), OR₂₃, —OC(O)R₂₀, —OC(O)OR₂₃, —OC(O)NHR₂₀, —OC(O)N(R₂₀)(R₂₁), —NHC(O)R₂₀, —NR₂OC(O)R₂₁, —NCO, —N₃, NHC(O)NHR₂₀, —NR₂OC(O)N(R₂₁)(R₂₂), —NHCOOR₂₃, —N(R₂₀)(R₂₁), —NR₂₀COOR₂₃, —N⁺(R₂₀)(R₂₁)(R₂₂) S⁺(R₂₀)(R₂₁) A⁻ or P⁺(R₂₀)(R₂₁)(R₂₂) A⁻; or the said alkyl and/or cycloalkyl both interrupted by and substituted by one or more heteroatomgroups (e.g. the ones defined above); or said aryl, heteroaryl and/or aralkyl substituted by 1 to 4 C₁-C₄alkyl;
R₂₀, R₂₁ and R₂₂ are independently C₁-C₁₈alkyl, C₂-C₁₈alkenyl, C₆-C₁₀aryl, C₅-C₈heteroaryl, C₇-C₁₁aralkyl or C₅-C₆cycloalkyl, preferably C₁-C₁₈alkyl; or said groups substituted by one or more F;
R₂₃ is H, NH₄, Li, Na, K or as defined for R₂₀, preferably H or C₁-C₁₈alkyl;
R₂₄ is C₁-C₁₈alkyl, C₆-C₁₀aryl, C₅-C₈heteroaryl, C₂-C₁₈alkenyl, C₂-C₁₈alkinyl, C₅-C₆cycloalkyl or glycidyl;
R₂₅ and R₂₆ are independently H, C₁-C₁₈alkyl, C₆-C₁₀aryl, C₂-C₁₈alkenyl, C₂-C₁₈alkinyl, C₅-C₆cycloalkyl or glycidyl;
R₂₇ is C₆-C₁₀aryl or —O—C₁-C₁₈ alkyl or —O—C₆-C₁₀aryl;
R₂₈ is H, —OH, C₁-C₁₈alkyl, C₆-C₁₀aryl, C₇-C₁₁aralkyl, C₅-C₆cycloalkyl, —O—C₁-C₁₈alkyl, —O—C₆-C₁₀aryl or —OQ, where Q is NH₄, Li, Na or K.

It is also possible that in the compounds of the present invention different kinds of groups -G- may be simultaneously present. In other words, some groups -G- may be present as nitroxide radicals >N—O., some as oxoammonium salts >N⁺═O, and some even as amines >N—H or hydroxylamines >N—OH.

A variety of positive electrodes may be employed in the disclosed lithium-ion cells. Some positive electrodes may be used with a wide range of compounds of formula (d1)-(d6), whereas other positive electrode materials having relatively high recharged potentials may be usable only with a smaller range of compounds of formula (d1)-(d6) having suitably higher oxidation potentials.

For example, the positive electrode comprises a compound selected from the group consisting of an organic radical (e.g. a nitroxyl radical), LiFePO₄, Li₂FeSiO₄, Li_{w}MnO₂, MnO₂, Li₄Ti₅O₁₂, LiMnP0₄, LiCo0₂, LiNi0₂, LiNi₁₋ₓCO_{y}Met_{z}0₂, LiMn_{0.5}Ni_{0.5}0₂, LiMn_{0.3}CO_{0.3}Ni_{0.3}0₂, LiFe0₂, LiMet_{0.5}Mn_{1.5}0₄, vanadium oxide, Li₁₊ₓMn_{2-z}Met_{y}0₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof,

whereby 0<m<0.5, 0<n<0.5, 0.3≦w≦0.4, 0<x<0.3, 0<z<0.5, 0<y<0.5, Met is Al, Mg, Ti, B, Ga, Si, Ni or Co, and X is S or F. Examples of such organic radicals are as outlined in EP1128453. More particularly, the organic radical can be as represented in EP1128453 as chemical formula (A1)-(A11), especially as chemical formula (A2) and (A7)-(A10), in particular as chemical formula (A7)-(A10). Further examples of such organic radicals are crosslinked polymers obtainable according to the process of WO-A-2007/115939 and compounds of formula (c1)-(c7), (d1)-(d7), (e1)-(e7) and the compounds in Table A, p. 23-57 of WO-A-2007/107468.

Powdered lithium (for example, LECTRO™ MAX stabilized lithium metal powder, from FMC Corp., Gastonia, N.C.) may be included in the positive electrode as formed. Lithium may also be incorporated into the negative electrode so that extractible lithium will be available for incorporation into the positive electrode during initial discharging. Some positive electrode materials may depending upon their structure or composition be charged at a number of voltages, and thus may be used as a positive electrode if an appropriate form and appropriate cell operating conditions are chosen. Electrodes made from LiFePO₄, Li₂FeSiO₄, LiₓMnO₂ (where x is about 0.3 to about 0.4, and made for example by heating a stoichiometric mixture of electrolytic manganese dioxide and LiOH to about 300 to about 400° C.) or MnO₂ (made for example by heat treatment of electrolytic manganese dioxide to about 350° C.) can provide cells having especially desirable performance characteristics when used with compounds of formula (d1)-(d6). The positive electrode may contain additives as will be familiar to those skilled in the art, for example, carbon black, flake graphite and the like. For instance, the positive electrode may be in any convenient form including foils, plates, rods, pastes or as a composite made by forming a coating of the positive electrode material on a conductive current collector or other suitable support.

For instance, the negative electrode comprises graphitic carbon, lithium metal, a lithium alloy (e.g. a Li/Sn alloy or a Li/Co alloy), amorphous material based on Sn and Co, or combinations thereof.

The graphitic carbon is, for example, that having a spacing between (002) crystallographic planes, d₀₀₂, of 3.45 Å>d₀₀₂>3.354 Å and existing in a form such as powder, flake, fiber or sphere (for example, mesocarbon microbead); the lithium alloy is for instance as described in U.S. Pat. No. 6,203,944 and in WO 00/103444, e.g. Li_{4/3}Ti_{5/3}O₄; Sn—Co-based amorphous negative electrodes (for example, the negative electrode in the NEXELION™ hybrid lithium ion battery from Sony Corp.); and combinations thereof. A negative electrode containing extractible lithium (for example, a lithium metal electrode, extractible lithium alloy electrode, or electrode containing powdered lithium) may be employed so that extractible lithium will be incorporated into the positive electrode during initial discharging. The negative electrode may contain additives as will be familiar to those skilled in the art, for example, carbon black. The negative electrode may be in any convenient form including foils, plates, rods, pastes or as a composite made by forming a coating of the negative electrode material on a conductive current collector or other suitable support.

The electrolyte (c) provides a charge-carrying pathway between the positive and negative electrodes. The electrolyte may include additionally to the components (i), (ii) and (iii) other additives that will be familiar to those skilled in the art. The electrolyte may be in any convenient form including liquids and gels.

Preferably, the lithium salt (i) is selected from the group consisting of LiPF₆, LiClO₄, LiBF₄, LiO₃SCF₃, LiN(C₂F₅SO₂)₂, LiC(CF₃SO₂)₃, LiC(C₂F₅SO₂)₃ or LiB(C₂O₄)₂, LiB(C₆H₅)₄, LiB(C₆F₅)₄, LiSbF₆, LiAsF₆, LiBr, LiBF₃C₂F₅, LiPF₃(CF₂CF₃)₃ and combinations thereof.

A variety of polar aprotic solvents (ii) may be employed in the electrolyte. Exemplary polar aprotic solvents (ii) are liquids or gels capable of solubilizing sufficient quantities of lithium salt (i) and a compound of formula (d1)-(d6) so that a suitable quantity of charge can be transported from the positive electrode to the negative electrode. Exemplary polar aprotic solvents (ii) can be used over a wide temperature range, for example, from about −30° C. to about 70° C. without freezing or boiling, and are stable in the electrochemical window within which the cell electrodes and the compound of formula (d1)-(d6) operate.

Preferably, the polar aprotic solvent (ii) is selected from the group consisting of ethylene carbonate, propylene carbonate, dimethyl carbonate, diethyl carbonate, methyl ethyl carbonate, γ-butyrolactone, tetrahydrofurane, dioxolane, sulfolane, dimethylformamide, dimethylacetamide, N-methyl-2-pyrrolidone, butylene carbonate, vinylene carbonate, fluoroethylene carbonate, fluoropropylene carbonate, methyl difluoroacetate, ethyl difluoroacetate, dimethoxyethane, bis(2-methoxyethyl)ether, and combinations thereof. The electrolyte also conveniently contains the dissolved component (iii), i.e. a compound of formula (d1)-(d6). The electrolyte can be formulated without component (iii), and incorporated into a cell whose positive or negative electrode contains dissolvable component (iii) that can dissolve into the electrolyte after cell assembly or during the first charge-discharge cycle, so that the electrolyte will contain dissolved component (iii) once the cell has been put into use.

For instance, component (iii) is a compound of formula (d1) or (d3), wherein

### X is O;

### R₁-R₄ are CH₃;

R₅ is C₁-C₁₈ alkyl, C₅-C₆cycloalkyl, —CO—R₁₆, —CON(R₁₆)(R₁₇), CO—OR₁₂, —PO(OR₁₂)(OR₁₃), —S(═O)₂R₁₂; or said groups substituted by one or more F; or said alkyl, alkenyl, alkinyl or cycloalkyl interrupted by one or more heteroatomgroup, preferably by O, NR₁₆; or the said alkyl, alkenyl, alkinyl or cycloalkyl substituted by one or more heteroatomgroup, preferably by —COOR₁₂, —CON(R₁₆)(R₁₇), OR₁₂, —OC(O)R₁₆, —OC(O)OR₁₂, —OC(O)N(R₁₆)(R₁₇), —NR₁₆C(O)R₁₇, —N₃, —NR₁₈C(O)N(R₁₆)(R₁₇), —N(R₁₆)(R₁₇), —NR₁₆COOR₁₂, more preferably by —O—CO—R₁₆, CO—OR₁₆ or OR₁₂; or the said alkyl and/or cycloalkyl both interrupted by and substituted by one or more heteroatomgroups (e.g. the ones defined above); or said aryl or aralkyl substituted by 1 to 4 C₁-C₄alkyl;
R₁₂ and R₁₃ are independently H, NH₄, Li, Na, K or as defined for R₁₆;
R₁₆, R₁₇ and R₁₈ are independently C₁-C₁₈alkyl, C₂-C₁₈alkenyl, C₆-C₁₀aryl, C₅-C₈heteroaryl, C₇-C₁₁aralkyl or C₅-C₆cycloalkyl; or said groups substituted by one or more F; or the said alkyl and/or cycloalkyl interrupted by one or more heteroatomgroup, preferably by O, NR₂₀, PR₂₀ or S, most preferably by O or NR₂₀; or said alkyl and/or cycloalkyl is substituted by one or more heteroatomgroup, preferably by Cl, —COOR₂₃, —CON(R₂₀)(R₂₁), OR₂₃, —OC(O)R₂₀, —OC(O)OR₂₃, —OC(O)N(R₂₀)(R₂₁), —NR₂OC(O)R₂₁, —NCO, —N₃, —NR₂OC(O)N(R₂₁)(R₂₂), —N(R₂₀)(R₂₁), —NR₂₀COOR₂₃; or the said alkyl and/or cycloalkyl both interrupted by and substituted by one or more heteroatomgroups (e.g. the ones defined above); or said aryl, heteroaryl and/or aralkyl substituted by 1 to 4 C₁-C₄alkyl;
R₂₀, R₂₁ and R₂₂ are independently C₁-C₁₈alkyl, C₂-C₁₈alkenyl, C₆-C₁₀aryl, C₅-C₈heteroaryl, C₇-C₁₁aralkyl or C₅-C₆cycloalkyl, preferably C₁-C₁₈alkyl; or said groups substituted by one or more F;
R₂₃ is H, NH₄, Li, Na, K or as defined for R₂₀, preferably H or C₁-C₁₈alkyl.

Preferred as component (iii) are compounds of formula (d1)-(d6), wherein

X is O for a compound of formula (d1);
X is S for a compound of formula (d2);
Y is —CH₂—O—CH₂—, —CH₂—S—CH₂—, —CH₂—S(═O)—CH₂—, —CH₂—S(═O)₂—CH₂—, —CH₂—NR₅—CH₂—,

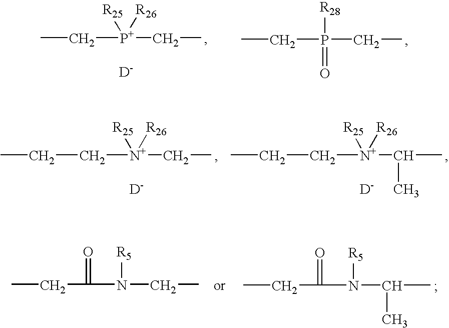

D⁻ is I⁻ or the anion of LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆F₅)₄, LiB(C₆F₅)₄, LiSbF₆, LiAsF₆, LiBr, LiBF₃C₂F₅ or LiPF₃(CF₂CF₃)₃, preferably I⁻ or ClO₄⁻;
R₁, R₂, R₃ and R₄ are independently C₁-C₁₈alkyl or C₆-C₁₀aryl; or said groups substituted by one or more F; or
R₁ and R₂ and/or R₃ and R₄ form together with the linking carbon atom a C₄-C₁₃cycloalkylbiradical which is unsubstituted or substituted by F;
R₅ is H, OH, C₁-C₁₈ alkyl, C₆-C₁₀aryl, C₇-C₁₁aralkyl, C₃-C₁₈alkenyl, C₃-C₁₈alkinyl, C₅-C₆cycloalkyl, glycidyl, —CO—R₁₆, —CO—NH—R₁₆, —CON(R₁₆)(R₁₇), —O—CO—R₁₆, —CO—OR₁₂, —(CH₂)_{q}COOR₁₂ or —PO(OR₁₂)(OR₁₃); or said groups substituted by one or more F; or said alkyl substituted by one or more OH;
R₆ and R₇ are independently an electron pair or ═O;
R₈ and R₉ are independently —CH₂O—CO—C₁-C₁₈alkyl, —CH₂—NH—CO—C₁-C₁₈alkyl or as defined for R₁;
or R₈ and R₉ form together with the linking carbon atom a

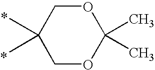

group;
R₁₀ and R₁₁ are independently H or CH₃;
R₁₂ and R₁₃ are independently H, NH₄, Li, Na, K or as defined for R₁₆; and
R₁₆ and R₁₇ are independently C₁-C₁₈alkyl, C₃-C₁₈alkenyl, C₆-C₁₀aryl or C₇-C₁₁aralkyl; or said groups substituted by one or more F;
R₂₅ and R₂₆ are independently H, C₁-C₁₈alkyl, C₆-C₁₀aryl, C₇-C₁₁aralkyl, C₂-C₁₈alkenyl, C₂-C₁₈alkinyl, C₅-C₆cycloalkyl or glycidyl;
R₂₈ is H, —OH, C₁-C₁₈alkyl, C₆-C₁₀aryl, C₇-C₁₁aralkyl, C₅-C₆cycloalkyl, —O—C₁-C₁₈alkyl, —O—C₆-C₁₀aryl or —OQ, where Q is NH₄, Li, Na or K;
and
q is an integer from 1 to 6.

More preferably, Y is —CH₂—S(═O)₂—CH₂—,

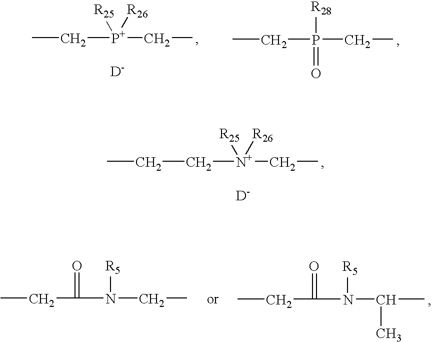

especially —CH₂—S(═O)₂—CH₂—;

### D⁻ is I⁻ or ClO₄⁻;

R₁, R₂, R₃ and R₄ are independently methyl, ethyl or propyl; or
R₁ and R₂ and/or R₃ and R₄ form together with the linking carbon atom a C₆-C₇cycloalkylbiradical;
R₅ is H, OH, C₁-C₈alkyl, phenyl, benzyl, C₃-C₆alkinyl, C₅-C₆cycloalkyl, glycidyl, —CO—R₁₆, —CO—C₁-C₅perfluoroalkyl, —CO—NH—R₁₆, —CO—OR₁₆ or —PO(OR₁₂)(OR₁₃); or said alkyl substituted by one OH;
R₆ and R₇ are independently an electron pair or ═O;
R₈ and R₉ are independently —CH₂O—CO—C₁-C₄alkyl, —CH₂—NH—CO—C₁-C₄alkyl or as defined for R₁;
or R₈ and R₉ form together with the linking carbon atom a

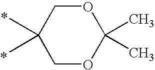

group;
R₁₀ and R₁₁ are independently H or CH₃;
R₁₂ and R₁₃ are independently H, NH₄, Li, Na, K or as defined for R₁₆, and
R₁₆ is C₁-C₈alkyl, C₃-C₆alkenyl, phenyl or benzyl;
R₂₅ and R₂₆ are C₁-C₈alkyl or phenyl; and
R₂₈ is phenyl;
with the proviso that R₅ can only be OH if R₆ and R₇ are both ═O.

Most preferably, Y is —CH₂—S(═O)₂—CH₂—,

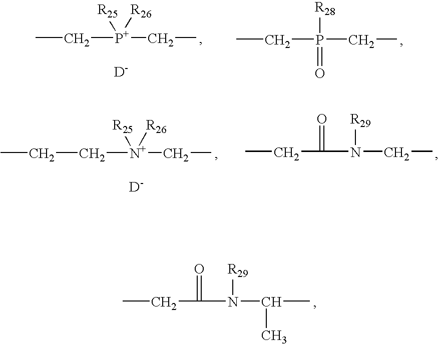

especially —CH₂—S(═O)₂—CH₂—;

### D⁻ is I⁻ or ClO₄⁻;

R₁, R₂, R₃ and R₄ are independently methyl, ethyl or propyl; or
R₁ and R₂ and/or R₃ and R₄ form together with the linking carbon atom a C₆-C₇cycloalkylbiradical;
R₅ is H, C₁-C₈alkyl, phenyl, benzyl, C₃-C₆alkinyl, glycidyl, —CO—R₁₆, —CO—NH—R₁₆, CO—OR₁₆ or —PO(OR₁₂)(OR₁₃); or said alkyl substituted by one OH;
R₆ and R₇ are independently an electron pair or ═O;
R₈ and R₉ are independently —CH₂O—CO—C₁-C₄alkyl or as defined for R₁;
R₁₀ and R₁₁ are independently H or CH₃;
R₁₂ and R₁₃ are independently as defined for R₁₆, preferably R₁₂ and R₁₃ are C₁-C₈alkyl, most preferably C₁-C₄alkyl;
R₁₆ is C₁-C₈alkyl, C₃-C₆alkenyl, phenyl or benzyl
R₂₅, R₂₆ are C₁-C₄alkyl or phenyl, preferably methyl or phenyl;
R₂₈ is phenyl; and
R₂₉ is H, C₁-C₄alkyl or —CO—O—O—C₁-C₈alkyl, preferably H, methyl or —CO—O-t-butyl.

Suitable as component (iii) are, for instance, the following nitroxides:

| No. | Structure |
|---|---|
| 1 | 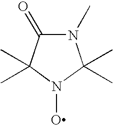 |
| 2 | 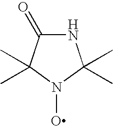 |
| 3 | 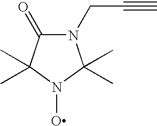 |
| 4 | 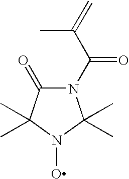 |
| 5 | 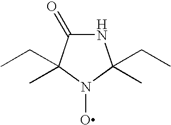 |
| 6 | 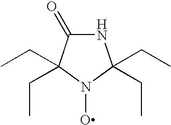 |
| 7 | 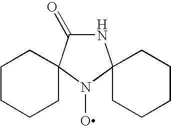 |
| 8 | 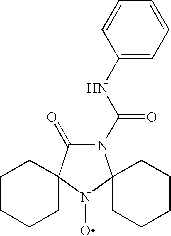 |
| 9 | 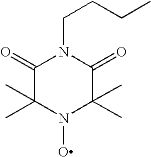 |
| 10 | 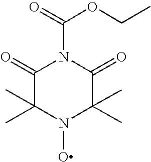 |
| 11 | 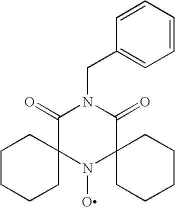 |
| 12 | 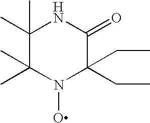 |
| 13 | 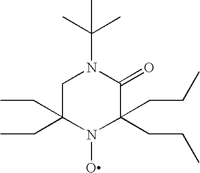 |
| 14 | 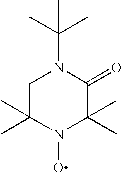 |
| 15 | 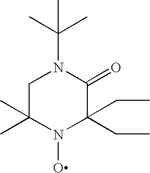 |
| 16 | 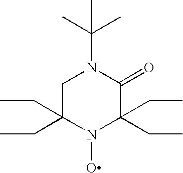 |
| 17 | 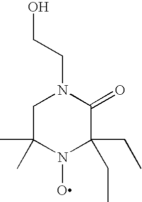 |
| 18 | 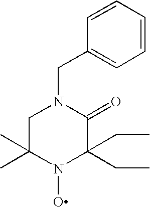 |
| 19 | 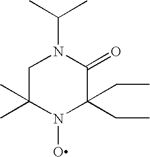 |
| 20 | 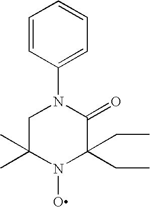 |
| 21 | 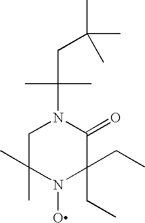 |
| 22 | 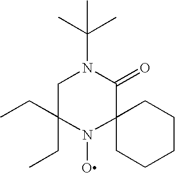 |
| 23 | 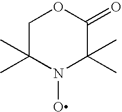 |
| 24 | 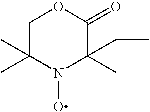 |
| 25 | 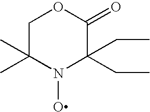 |
| 26 | 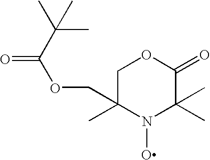 |
| 27 | 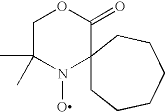 |
| 28 | 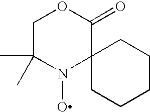 |
| 29 | 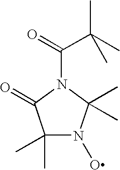 |
| 30 | 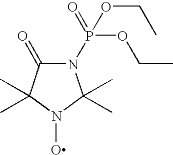 |
| 31 | 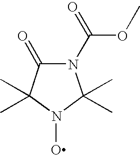 |
| 32 | 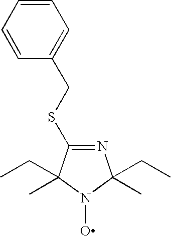 |
| 33 | 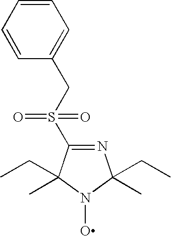 |
| 34 | 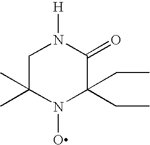 |
| 35 | 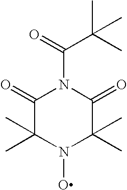 |
| 36 | 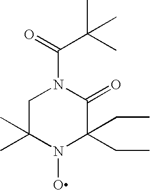 |
| 37 | 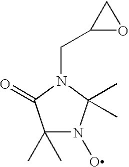 |
| 38 | 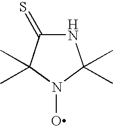 |
| 39 | 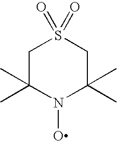 |
| 40 | 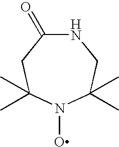 |
| 41 | 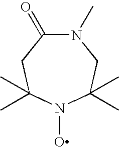 |
| 42 | 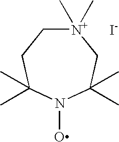 |
| 43 | 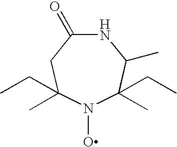 |
| 44 | 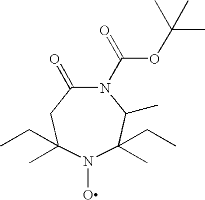 |
| 45 | 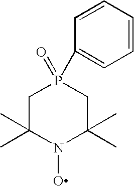 |
| 46 | 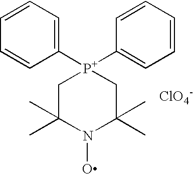 |

Preferably, the compound (iii) is dissolved in the electrolyte.

A preferred embodiment is a rechargeable lithium-ion cell comprising:

(a) a positive electrode (e.g. having a recharged potential) comprising a compound selected from the groups consisting of LiFePO₄, Li₂FeSiO₄, Li_{w}MnO₂, MnO₂, Li₄Ti₅O₁₂, LiMnP0₄, LiCo0₂, LiNi0₂, LiNi₁₋ₓCO_{y}Met_{z}0₂, LiMn_{0.5}Ni_{0.5}0₂, LiMn_{0.3}CO_{0.3}Ni_{0.3}0₂, LiFe0₂, LiMet_{0.5}Mn_{1.5}0₄, vanadium oxide, Li₁₊ₓMn_{2-z}Met_{y}0₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃, and combinations thereof,
whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F;
(b) a negative electrode comprising graphitic carbon, lithium metal, a lithium alloy (e.g. a Li/Sn alloy or a Li/Co alloy), amorphous material based on Sn and Co, or combinations thereof; and
(c) an electrolyte comprising:
(i) a lithium salt selected from the group consisting of LiPF₆, LiClO₄, LiBF₄, LiO₃SCF₃, LiN(C₂F₅SO₂)₂, LiC(CF₃SO₂)₃, LiC(C₂F₅SO₂)₃ or LiB(C₂O₄)₂, LiB(C₆H₅)₄, LiB(C₆F₅)₄, LiSbF₆, LiAsF₆, LiBr, LiBF₃C₂F₅, LiPF₃(CF₂CF₃)₃, and combination thereof;
(ii) a polar aprotic solvent selected from the group consisting of ethylene carbonate, propylene carbonate, dimethyl carbonate, diethyl carbonate, methyl ethyl carbonate, γ-butyrolactone, tetrahydrofurane, dioxolane, sulfolane, dimethylformamide, dimethylacetamide, N-methyl-2-pyrrolidone, butylene carbonate, vinylene carbonate, fluoroethylene carbonate, fluoropropylene carbonate, methyl difluoroacetate, ethyl difluoroacetate, dimethoxyethane, bis(2-methoxyethyl)ether and combinations thereof; and
(iii) at least one compound selected from the group consisting of formula (d1)-(d6) as defined above dissolved in the electrolyte.

For instance, the amount of (i) is 1-50%, preferably 5-30%, most preferably 10-25% by weight of (ii).

For example, the amount of (iii) is 0.1-50%, preferably 1-20%, most preferably 2-10% by weight of (ii).

An embodiment is a method for manufacturing a rechargeable lithium-ion sealed cell comprising the steps of assembling in any order and enclosing in a suitable case:

(a) a positive electrode (e.g. having a recharged potential);
(b) a negative electrode; and
(c) an electrolyte comprising
(i) a lithium salt,
(ii) a polar aprotic solvent and
(iii) at least one compound selected from the group consisting of formula (d1)-(d6) as defined above.

The arrangement of the lithium-ion cell can be as described in WO 2006/124389.

The described lithium-ion cells may include a porous cell separator located between the positive and negative electrodes and through which charge-carrying species (including the compound (iii)) may pass. Suitable separators will be familiar to those skilled in the art. The disclosed cells may be sealed in a suitable case, for example, in mating cylindrical metal shells such as in a coin-type cell, in an elongated cylindrical AAA, AA, C or D cell casing or in a replaceable battery pack as will be familiar to those skilled in the art. The described cells may be used in a variety of devices, including portable computers, tablet displays, personal digital assistants, mobile telephones, motorized devices (e.g, personal or household appliances and vehicles), instruments, illumination devices (for example, flashlights) and heating devices. The disclosed cells may have particular utility in low-cost mass market electrical and electronic devices such as flashlights, radios, CD players and the like, which heretofore have usually been powered by non-rechargeable batteries such as alkaline cells. Further details regarding the construction and use of rechargeable lithium-ion cells will be familiar to those skilled in the art.

An embodiment is a rechargeable lithium-ion cell, wherein the compound (iii) is a cyclable redox chemical shuttle which is dissolved in or is dissolvable in the electrolyte and having an oxidation potential above the recharged potential of the positive electrode.

When an attempt is made to charge the cell above the oxidation potential of the cyclable redox chemical shuttle (compound (iii) i.e. the compound of formula (d1)-(d6)), the oxidized cyclable redox chemical shuttles carry a charge quantity corresponding to the applied charging current to the negative electrode, thus preventing cell overcharge.

The compound (iii) has usually an oxidation potential that is higher (i.e. more positive) than the recharged potential of the positive electrode. For instance, the oxidation potential of the compound (iii) is just slightly higher than the recharged potential of the positive electrode and below the potential at which irreversible cell damage might occur, and desirably below the potential at which excessive cell heating or outgassing might occur.

Preferred are compounds (iii) which have an oxidation potential from 0.3 V to 5 V, preferably from 0.3 to 0.6 V, above the recharged potential of the positive electrode.

For example, compound (iii) provides overcharge protection after at least 30 charge-discharge cycles, preferably after at least 80 charge-discharge cycles, in particular after at least 100 charge-discharge cycles, at a charging voltage sufficient to oxidize compound (iii), wherein G is

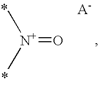

and at an overcharge charge flow equivalent to 100% of the cell capacity during each cycle.

Mixtures of two or more compounds (iii) having different electrochemical potentials may also be employed. For example, a first compound (iii) operative at a lower voltage and a second compound (iii) operative at a higher voltage may both be employed in a single cell. If after many charge/discharge cycles the first compound (iii) degrades and loses its effectiveness, the second compound (iii) (which would not meanwhile have been oxidized while the first compound (iii) was operative) could take over and provide a further (albeit higher E_{cv}) margin of safety against overcharge damage. The compound (iii) can also provide overdischarge protection to a cell or to a battery of series-connected cells; such overdischarge protection can be obtained analogueously to WO 2005/099025.

An embodiment is a method for recharging a lithium-ion cell while chemically limiting cell damage due to overcharging comprising supplying charging current across a positive and a negative electrode of a lithium-ion rechargeable cell containing an electrolyte (c) comprising a lithium salt (i), a polar aprotic solvent (ii) and a cyclable redox chemical shuttle comprising a compound (iii) as defined above dissolved in the electrolyte and having an oxidation potential above the recharged potential of the positive electrode.

Preferred is the use of a compound (iii) as defined above as a cyclable redox chemical shuttle in a rechargeable lithium-ion cell.

An embodiment is a rechargeable lithium-ion cell, wherein the compound (iii) is a molecular redox shuttle for redox targeting.

For instance, the molecular redox shuttle (i.e. compound (iii)) is dissolved in the electrolyte of the positive or negative electrode, especially in the electrolyte of the positive electrode.

During charging, the molecular redox shuttle (S) is oxidized at the current collector to the cation of molecular redox shuttle (S⁺)

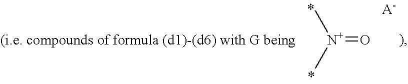

which delivers the charge to the electrode by bulk diffusion. S⁺ will be reduced back to S

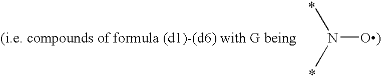

by hole injection in the electrode particles. During the discharging process, S⁺ is reduced at the current collector to S, which in turn delivers electrons to the oxidized electrode particles. The advantage of using a freely diffusing molecular redox shuttle is that it allows charge transport to proceed at a fast rate, thus the power output of the battery is huge. Usually, the active electrode materials are in electronic contact with the current collector. The electrode materials are normally prepared with conducting additives to form an electrode sheet that is attached to a metal support. For instance, in the presence of the described molecular redox shuttle, no or only a lower amount of conducting additives are needed and the energy density of the electrodes is greatly improved.

Also preferred is the use of a compound (iii) as defined above as a molecular redox shuttle for redox targeting.

Another embodiment is a compound (d1)-(d6) as defined above,

wherein

### G is

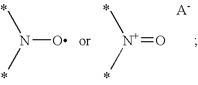

when G is

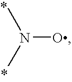

the compound is of formula (d1), (d3) or (d4) and
R₅ is —CO—R₁₆, —CO—NH—R₁₆, —CON(R₁₆)(R₁₇), CO—OR₁₆, —O—CO—R₁₆, —(CH₂)_{q}COOR₁₂, —PO(OR₁₂)(OR₁₃), —S(═O)₂OR₁₂, —SR₁₂, —S(═O)R₁₂, —S(═O)₂R₁₂, —S—OR₁₂, —S(═O)—OR₁₂, —SiR₁₆R₁₇R₁₈, —CN or -halogen; preferably R₅ is —CO—R₁₆, —CO—NH—R₁₆, CO—OR₁₆, —(CH₂)_{q}COOR₁₂, —PO(OR₁₂)(OR₁₃), —S(═O)₂OR₁₂, —SiR₁₆R₁₇R₁₈, —CN or -halogen; most preferably R₅ is —CO—R₁₆, —CO—NH—R₁₆, CO—OR₁₆, —PO(OR₁₂)(OR₁₃), —S(═O)₂OR₁₂, —SiR₁₆R₁₇R₁₈, —CN or -halogen;
q is an integer from 1 to 6;
with the proviso
that for compounds of formula (d1), R₅ is —PO(OR₁₂)(OR₁₃), —S(═O)₂OR₁₂, —SR₁₂, —S(═O)R₁₂, —S(═O)₂R₁₂, —S—OR₁₂, —S(═O)—OR₁₂ or —SiR₁₆R₁₇R₁₈; and that the compounds

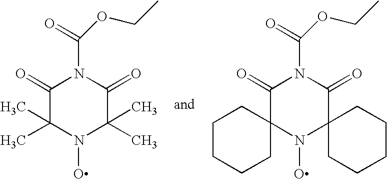

are excluded.

For instance, the same preferences apply to these compound as to the compounds (iii) in a rechargeable cell.

The precursor compounds of the compounds of formula (d1)-(d6) are essentially known and partially commercially available. All of them can be prepared by known processes. Their preparation is disclosed, for example, in: A. Khalaj et al., Monatshefte für Chemie, 1997, 128, 395-398; S. D. Worley et al., Biotechnol. Prog., 1991, 7, 60-66; T. Toda et al., Bull. Chem. Soc. Jap., 1972, 45, 557-561.

The oxidation of the aminic precursors into nitroxides may be carried out in analogy to the oxidation of 4-hydroxy-2,2,6,6-tetramethylpiperidine described in U.S. Pat. No. 5,654,434 with hydrogen peroxide. Another also suitable oxidation process is described in WO 00/40550 using peracetic acid.

An exhaustive description of the nitroxide chemistry can be found, for example, in L. B. Volodarsky, V. A. Reznikov, V. I. Ovcharenko.: “Synthetic Chemistry of Stable Nitroxides”, CRC Press, 1994.

The compounds with Y being —CH₂—O—CH₂— can be prepared via cyclodehydration of the corresponding aminodiols as described for example by: J. T. Lai: Synthesis 122-123, (1984).

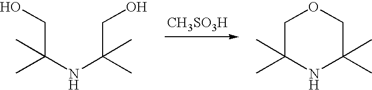

The compounds with Y being —CH₂—S(═O)₂—CH₂— can be prepared via cyclization of dimethallylsulfone with ammonia as described in DE 2 351 865. This patent reports also the preparation of the corresponding nitroxides.

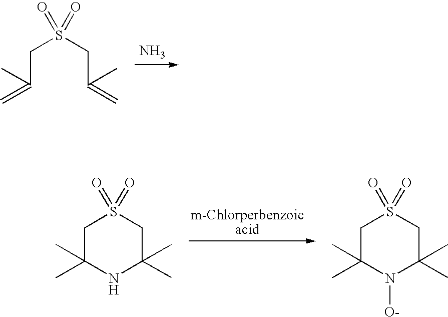

The compounds with Y being —CH₂—S(═O)—CH₂— can be prepared via reductive elimination of one oxygen atom as described for example by: Still, Ian W. J.; Szilagyi, Sandor: Synthetic Communications (1979), 9(10), 923-30.

The compounds with Y being —CH₂—S—CH₂— can be prepared via reductive elimination of two oxygen atoms as described for example by: Akgun, Eyup; Mahmood, Khalid; Mathis, Chester: Journal of the Chemical Society, Chemical Communications (1994), (6), 761-2.

The compounds with Y being

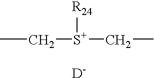

can be prepared via diverse methods well-known for the preparation of sulfonium salts.

The compounds with Y being

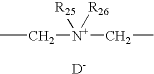

or —CH₂—NR₅—CH₂— can be prepared via reduction of the corresponding piperazindiones or piperazinones with LiAlH₄ as described for example by: Kaliska, Viera; Toma, Stefan; Lesko, Jan.: Collection of Czechoslovak Chemical Communications (1987), 52(9), 2266-73.

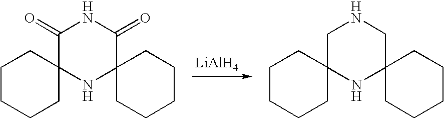

The obtained compounds can be further functionalized on the N-atom via alkylations, acylations etc which are well known standard reactions.

The compounds with Y being

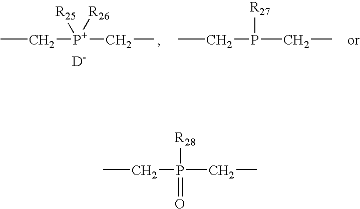

can be prepared via cyclization of dimethallylphosphonium salts, optionally followed by hydrolytic removal of one group from phosphorus, as described by Skolimowski, J.; Skowronski, R.; Simalty, M.: Tetrahedron Letters 4833-4 (1974).

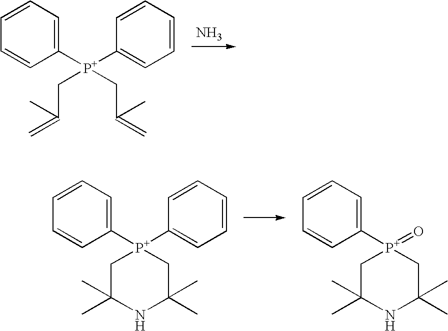

Compounds with Z═CH₂—CH₂— or —CH₂—CO— and Q=—CH₂— can be prepared as described in or in analogy to the work by Ramasseul, R.; Rassat A.; Rey, P.: Tetrahedron Letters 839 (1975).

Compounds with Y being

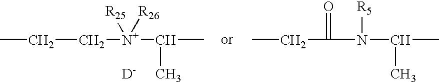

can be prepared as described in U.S. Pat. No. 6,664,353 B2.

Preparation of Compounds with Y being

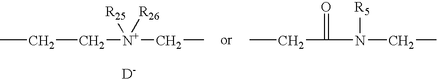

can be prepared as described by Rozantsev, E. G.; Chudinov, A. V.; Sholle, V. D.: lzvestiya Akademii Nauk SSSR, Seriya Khimicheskaya (1980), (9), 2114-17.

The methods described in WO 2004/031150 can be used for the preparation of oxoammonium salts.

The term alkyl comprises within the given limits of carbon atoms, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, 2-methylheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl or dodecyl.

Alkyl that is interrupted by one or more heteroatomgroups comprises at least two carbon atoms.

Alkenyl and alkinyl that are interrupted by one or more heteroatomgroups comprise at least three carbon atoms.

For instance, heteroaryl contains 1 or 2 heteroatoms, especially O, N, P, S or combinations thereof.

Examples of heteroaryl are furane, pyrrol, thiophene, pyridine, imidazol, oxazol, thiazol, triazol, pyridine, pyridazine, pyrimidine or pyrazine.

Examples of alkenyl are within the given limits of carbon atoms vinyl, allyl, and the branched and unbranched isomers of butenyl, pentenyl, hexenyl, heptenyl, octenyl, nonenyl, decenyl, undecenyl and dodecenyl. The term alkenyl also comprises residues with more than one double bond that may be conjugated or non-conjugated, for example may comprise one double bond.

Examples of alkinyl are within the given limits of carbon atoms ethinyl, propinyl and the branched and unbranched isomers of butinyl, pentinyl, hexinyl, heptinyl, octinyl, noninyl, decinyl, undecinyl and dodecinyl. The term alkinyl also comprises residues with more than one triple bond that may be conjugated or non-conjugated and residues with at least one triple bond and at least one double bond, for example comprises residues with one triple bond.

Some examples of cycloalkyl are cyclopentyl, cyclohexyl, methylcyclopentyl or dimethylcyclopentyl, especially cyclopentyl or cyclohexyl, in particular cyclohexyl.

Some examples of cycloalkylbiradical are 1,1-cyclopentylbiradical, 1,1-cyclohexylbiradical or 1,1-cycloheptylbiradical, especially 1,1-cyclohexylbiradical or 1,1-cycloheptylbiradical.

Aryl is for example phenyl.

Aralkyl is for instance benzyl or α,α-dimethylbenzyl.

The term halogen may comprise fluorine, chlorine, bromine and iodine; for example halogen is fluorine.

Groups substituted by one or more F can be perfluorinated (in particular all hydrogen atoms of said group are replaced by F).

Percentages are weight-% and ratios are ratio by weight unless otherwise stated.

### ABBREVIATIONS

cmpd compound
CV cyclic voltammetry
DMF dimethylformamide
EDTA ethylenediaminetetraacetic acid
MS mass spectrometry
NMR nuclear magnetic resonance
sat'd saturated
satd saturated
TEMPO 2,2,6,6-tetramethylpiperidin-N-oxyl
THF tetrahydrofuran

### SYNTHESIS EXAMPLES

### Example 1

### Cmpd 1

Hydrogen peroxide (aqueous, 30%, 2.5 g, 22 mmol) is slowly added to a solution of 2,2,3,5,5-pentamethyl-imidazolidin-4-one (1.85 g, 10 mmol) in acetic acid (15 ml) containing EDTA (0.0497 g, 0.17 mmol) and Na₂WO₄×2H₂O (0.0495 g, 0.15 mmol) and the resulting pale yellow suspension stirred overnight at room temperature (25° C.). Additional hydrogen peroxide (2.4 g, 21 mmol) is fed in and the orange solution stirred for another 2 days. The reaction mixture is brought to pH 7 (aqueous NaOH, 30%) and the resulting orange suspension extracted with CH₂Cl₂ (2×40 ml). The organic phase is brine-washed, dried over MgSO₄ and the solvent distilled off on a rotary evaporator to leave a reddish oil that solidified upon standing. Purification by chromatography (silica gel, hexane/ethylacetate 4/6) gives 0.4 g of the title compound as orange crystals, mp. 67-69° C. MS: for C₈H₁₅N₂O₂ (171.22) found M⁺=171.

### Intermediates

### A) 2,2,5,5-Tetramethyl-imidazolidin-4-one

Prepared as described in EP1283240 (2003; to D. Lazzari et al, Ciba Specialty Chemicals Holding Inc.; CAN 138:154404).

### B) 2,2,3,5,5-Pentamethyl-imidazolidin-4-one

Methyl iodide (3.6 g, 25 mmol) is slowly added to an ice-cooled suspension of 2,2,5,5-tetramethyl-imidazolidin-4-one (3.55 g, 25 mmol) in toluene (10 ml) containing potassium tert-butoxide (2.9 g, 25 mmol). The ice-bath is removed and the reaction mixture stirred overnight. Filtration and evaporation of the solvent on a rotary evaporator leaves a yellowish oil. Fractional short-path vacuum distillation using a Kugelrohr-oven affords 2 g of the title compound as a colourless liquid. MS: for C₈H₁₆N₂O (156.23) found M⁺=156. ¹H-NMR (300 MHz, CDCl₃), δ (ppm) 2.81 (s, 3H), 1.78 (br s, 1H), 1.39 (s, 6H), 1.33 (s, 6H).

### Example 2

### Cmpd 2

prepared as described in: Toda, Toshimasa; Morimura, Syoji; Mori, Eiko; Horiuchi, Hideo; Murayama, Keisuke: Bulletin of the Chemical Society of Japan (1971), 44(12), 3445-50.

### Example 3

### Cmpd 3

2,2,5,5-Tetramethyl-imidazolidin-4-one-1-N-oxyl (Cmpd 2) (23.6 g, 0.15 mol) is dissolved in dry DMF (100 ml) and sodium hydride (0.157 mol, 6.9 g of 55% dispersion in parrafin oil) is slowly added. The mixture is stirred at 40° C. for 2 h and then cooled to 3° C. Propargyl bromide (19.6 g, 0.165 mol) is then added over 45 minutes while keeping the temperature at 3-8° C. The mixture is stirred for additional 15 h at room temperature and then diluted with water (1000 ml). The solid is filtered off and chromatographed on silica gel column with dichloromethane-ethyl acetate (4:1) to afford 22.8 g of red crystals, mp. 119-121° C.

### Example 4

### Cmpd 4

2,2,5,5-Tetramethyl-imidazolidin-4-one-1-N-oxyl (Cmpd 2) (1.73 g, 0.011 mol), triethylamine (1.7 ml, 0.012 mol) and 4-dimethylaminopyridine (67 mg) are dissolved in dichloromethane (12 ml). Methacryloyl chloride (1.27 g, 0.012 mol) is added slowly to the stirred solution while keeping the temperature at 3-8° C. The mixture is stirred for 2 h at room temperature, then washed with water (3×5 ml) and evaporated. The solid residue is recrystallized from methanol to afford 2.08 g of red crystals, mp. 94-96° C.

### Example 5

### Cmpd 5

prepared as described in: Toda, Toshimasa; Morimura, Syoji; Mori, Eiko; Horiuchi, Hideo; Murayama, Keisuke: Bulletin of the Chemical Society of Japan (1971), 44(12), 3445-50.

### Examples 6, 12, 13, 15, 16, 17, 18, 19, 20, 21, 22, 24, 25, 26

### Cmpds 6, 12, 13, 15, 16, 17, 18, 19, 20, 21, 22, 24, 25, 26

prepared as described in: Nesvadba, P., Kramer, A., Zink, M.-O.: U.S. Pat. No. 6,479,608 B1, (2002), cmpd 6 (Example A 4 of U.S. Pat. No. 6,479,608 B1), cmpd 12 (Example B 34 of U.S. Pat. No. 6,479,608 B1), cmpd 13 (Example B 68 of U.S. Pat. No. 6,479,608 B1), cmpd 15 (Example B 30 of U.S. Pat. No. 6,479,608 B1), cmpd 16 (Example B 57 of U.S. Pat. No. 6,479,608 B1), cmpd 17 (Example B 77 of U.S. Pat. No. 6,479,608 B1), cmpd 18 (Example B 37 of U.S. Pat. No. 6,479,608 B1), cmpd 19 (Example B 26 of U.S. Pat. No. 6,479,608 B1), cmpd 20 (Example B 88 of U.S. Pat. No. 6,479,608 B1), cmpd 21 (Example B 74 of U.S. Pat. No. 6,479,608 B1), cmpd 22 (Example B 62 of U.S. Pat. No. 6,479,608 B1), cmpd 24 (Example B 1 of U.S. Pat. No. 6,479,608 B1), cmpd 25 (Example B 5 of U.S. Pat. No. 6,479,608 B1), cmpd 26 (Example B11 of U.S. Pat. No. 6,479,608 B1)

### Example 7

### Cmpd 7

prepared as described in: Toda, Toshimasa; Morimura, Syoji; Mori, Eiko; Horiuchi, Hideo; Murayama, Keisuke: Bulletin of the Chemical Society of Japan (1971), 44(12), 3445-50.

### Example 8

### Cmpd 8

prepared as described in: Chalmers, Alexander M.: (Ciba-Geigy), Ger. Offen. (1975), DE 2500313, Example 14.

### Example 9

### Cmpd 9

prepared in analogy to Example 7 of: Ramey, Chester E.; Luzzi, John J. U.S. Pat. No. 3,936,456 (1976). Red crystals, mp.=52-54° C.

### Example 10

### Cmpd 10

prepared as described in: Yoshioka, Takao; Mori, Eiko; Murayama, Keisuke: Bulletin of the Chemical Society of Japan (1972), 45(6), 1855-60.

### Example 11

### Cmpd 11

prepared as described in: Yoshioka, Takao; Mori, Eiko; Murayama, Keisuke: Bulletin of the Chemical Society of Japan (1972), 45(6), 1855-60.

### Example 14

### Cmpd 14

prepared as described in: Lai, John T. Synthesis (1981), (1), 40-2.

### Example 23

### Cmpd 23

prepared as described in: Lai, John Ta-yuan; Filla, Deborah S. WO 2001023435 A1, Example 2.

### Examples 27, 28

### Cmpds 27, 28

prepared as described in: Lai, John Ta-yuan; Masler, William F.; Nicholas, Paul Peter; Pourahmady, Naser; Puts, Rutger D.; Tahiliani, Shonali, EP 869137 A1, Examples 5 and 6.

### Example 29

### Cmpd 29

### 3-(2,2-Dimethyl-propionyl)-2,2,5,5-tetramethyl-imidazolidin-4-one-1-N-oxyl

2,2,5,5-Tetramethyl-imidazolidin-4-one-1-N-oxyl (Cmpd 2) (1.73 g, 0.011 mol), triethylamine (1.7 ml, 0.012 mol) and 4-dimethylaminopyridine (67 mg) are dissolved in dichloromethane (12 ml). Pivaloyl chloride (1.46 g, 0.012 mol) is added slowly to the stirred solution while keeping the temperature at 3-8° C. The mixture is stirred for 2 h at room temperature, then washed with water (3×5 ml) and evaporated. The solid residue is chromatographed on silica gel (hexane-ethyl acetate 3:1) and recrystallized from hexane to afford 1.85 g of red crystals, mp. 69-71° C. MS: for C₁₂H₂₁N₂O₃ (241.3) found M⁺=241.

### Example 30

### Cmpd 30

### (2,2,4,4-Tetramethyl-5-oxo-imidazolidin-3-N-oxy-1-yl)-phosphonic acid diethyl ester

2,2,5,5-Tetramethyl-imidazolidin-4-one-1-N-oxyl (Cmpd 2) (1.60 g, 0.01 mol) is dissolved in dimethyl formamide (13 ml). Sodium hydride (0.48 g, 0.011 mol, 55% in parrafin oil) is then added and the mixture is stirred 60 minutes at 50° C. The mixture is then cooled to 2° C. and diethyl chlorophosphate (1.97 g, 0.011 mol) is added during 5 minutes. Water (150 ml) is added after 17 h stirring at room temperature and the mixture is extracted with methylene chloride (3×30 ml). The combined extracts are evaporated and the residue is chromatographed on silica gel (hexane-ethyl acetate 1:1) and recrystallized from dichloromethane-hexane to afford 2.1 g of red crystals, mp. 78-80° C. MS: for C₁₁H₂₂N₂O₅P (293.3) found M⁺=293.

### Example 31

### Cmpd 31

### 2,2,4,4-Tetramethyl-5-oxo-imidazolidine-3-N-oxyl-1-carboxylic acid methyl ester

2,2,5,5-Tetramethyl-imidazolidin-4-one-1-N-oxyl (Cmpd 2) (1.73 g, 0.011 mol), triethylamine (1.7 ml, 0.012 mol) and 4-dimethylaminopyridine (67 mg) are dissolved in dichloromethane (15 ml). Methyl chloroformiate (1.14 g, 0.012 mol) is added slowly to the stirred solution while keeping the temperature at 3-8° C. The mixture is stirred for 4 h at room temperature. More 4-dimethylaminopyridine (50 mg), triethylamine (0.85 ml) and methyl chloroformiate (0.5 ml) is then added and the mixture is stirred for additional 3 h, then washed with water (3×10 ml) and evaporated. The solid residue is chromatographed on silica gel (methylene chloride-ethyl acetate 25:1) and recrystallized from dichloromethane-hexane to afford 1.4 g of red crystals, mp. 82-86° C. MS: for C₉H₁₅N₂O₄ (215.2) found M⁺=215.

### Example 32

### Cmpd 32

### 4-Benzylsulfanyl-2,5-diethyl-2,5-dimethyl-2,5-dihydro-1H-imidazole-1-N-oxyl

### A) 4-Benzylsulfanyl-2,5-diethyl-2,5-dimethyl-2,5-dihydro-1H-imidazole

2,2,5,5-Tetramethyl-imidazolidin-4-thione (33.5 g, 0.18 mol), acetone (300 ml), potassium carbonate (26.1 g, 0.189 mol) and benzyl bromide (32.3 g, 0.189 mol) are stirred under reflux for 5 h. The solids are then filtered off and washed with acetone. The filtrate is evaporated to afford 50 g of the title compound as viscous, yellow oil.

### B) Oxidation

4-Benzylsulfanyl-2,5-diethyl-2,5-dimethyl-2,5-dihydro-1H-imidazole (48.1 g, 0.174 mol) is dissolved in ethyl acetate (400 ml). m-Chloroperbenzoic acid (64.35 g, 0.26 mol, 70% content) is then added during 30 minutes while keeping the temperature at 10-15° C. The mixture is stirred for 2 h at room temperature and additional 20 g m-chloroperbenzoic acid are added. After 2 h of stirring another 20 g of m-chloroperbenzoic acid are added and the mixture is stirred for 16 h at room temperature, then washed with 1M-NaHCO₃ (3×300 ml) and evaporated. The residue is chromatographed on silica gel with hexane-ethyl acetate (9:1 to 6:1) to afford 9.5 g of the title compound as a red oil. For C₁₆H₂₃N₂OS (291.44) calculated C, 65.94%; H, 7.95%; N, 9.61%. found C, 65.89%; H, 7.95%; N, 9.53%.

### Example 33

### Cmpd 33

### 2,5-Diethyl-2,5-dimethyl-4-phenylmethanesulfonyl-2,5-dihydro-1H-imidazole-1-N-oxyl

This compound is obtained by recrystallization from hexane of the polar fractions obtained during the chromatographic purification of Cmpd 32 as an orange solid, 8.1 g, 66-72° C. MS: for C₁₆H₂₃N₂O₃S (323.4) found M⁺=323.

### Example 34

### Cmpd 34

### 3,3-Diethyl-5,5-dimethyl-piperazin-2-one-4-N-oxyl

### A) 3,3-Diethyl-5,5-dimethyl-piperazin-2-one

1-t-Butyl-3,3-Diethyl-5,5-dimethyl-piperazin-2-one (315.7 g, 1.3 mol, prepared as described in Nesvadba, Peter; Kramer, Andreas; Zink, Marie-odile. Ger. Offen. (2000), DE-A-19949352) is slowly added to hydrochloric acid (316 ml, 37%) and the mixture is refluxed for 24 h and then poured into a cold solution of NaOH (151 g, 3.775 mol) in 500 ml water. The organic layer (t-butylchloride) is discarded and the aqueous layer is extracted with t-butyl-methyl ether (5×100 ml). The combined extracts are dried over MgSO₄ and evaporated to afford crude title compound (256 g) as a yellow liquid.

### B) Oxidation

To a solution of 3,3-diethyl-5,5-dimethyl-piperazin-2-one (9.21 g, 0.05 mol) in ethyl acetate (25 ml) is slowly added peracetic acid (15.8 g, 0.083 mol, 40% in acetic acid) and the mixture is stirred for 8 h at room temperature. Water (100 ml) is then added and the mixture is extracted with t-butyl-methyl ether (6×35 ml). The extracts are washed with 5% NaOH (100 ml), dried over MgSO₄ and evaporated. The residue is recrystallized from toluene-hexane to afford 6.56 g of the title compound as yellow crystals, mp. 126-129° C. For C₁₀H₁₉N₂O₂ (199.27) calculated C, 60.27%; H, 9.61%; N, 14.05%. found C, 60.37%; H, 9.67%; N, 13.93%.

### Example 35

### Cmpd 35

### 1-(2,2-Dimethyl-propionyl)-3,3,5,5-tetramethyl-piperazine-2,6-dione-4-N-oxyl

### A) 1-(2,2-Dimethyl-propionyl)-3,3,5,5-tetramethyl-piperazine-2,6-dione

3,3,5,5-tetramethyl-piperazine-2,6-dione (1.7 g, 0.01 mol, prepared according to Bulletin of the Chemical Society of Japan (1972), 45(6), 1855), triethylamine (1.6 ml, 0.011 mol) and 4-dimethylaminopyridine (55 mg) is dissolved in methylene chloride (20 ml). Then, pivaloyl chloride (1.33 g, 0.011 mol) is added during 3 minutes and the mixture is stirred for 20 h at room temperature. Methylene chloride (50 ml) and water (50 ml) is then added, the organic layer is separated and chromatographed on silica gel with dichloromethane-ethyl acetate (4:1) to afford 2.42 g of the title compound as a colorless solid, mp. 100-102° C. MS: for C₁₆H₂₃N₂O₃S (323.4) found M⁺=323.

### B) Oxidation

To a stirred mixture of 1-(2,2-dimethyl-propionyl)-3,3,5,5-tetramethyl-piperazine-2,6-dione (1.75 g, 6.88 mmol), NaHCO₃ (1.8 g, 21.4 mmol), methylene chloride (20 ml) and water (3 ml) is slowly added peracetic acid (2.1 g, 11 mmol, 40% in acetic acid) and the mixture is stirred for 17 h at room temperature. Additional 0.33 g of peracetic acid are added and the stirring is continued for 2 h. The organic layer is then separated, washed with 2 M Na₂CO₃ (2×10 ml) and evaporated. The residue is chromatographed on silica gel with dichloromethane and crystallized from hexane to afford 0.78 g of the title compound as red crystals, mp. 115-117° C. MS: for C₁₃H₂₁N₂O₄ (269.3) found M⁺=269.

### Example 36

### Cmpd 36

### 1-(2,2-Dimethyl-propionyl)-3,3-diethyl-5,5-dimethyl-piperazin-2-one-4-N-oxyl

3,3-Diethyl-5,5-dimethyl-piperazin-2-one-4-N-oxyl (Cmpd 34) (1.99 g, 0.01 mol), triethylamine (1.6 ml, 0.011 mol) and 4-dimethylaminopyridine (56 mg) are dissolved in dichloromethane (12 ml). Pivaloyl chloride (1.32 g, 0.011 mol) is added slowly to the stirred solution while keeping the temperature at 3-8° C. The mixture is then stirred for 3 h at room temperature, then washed with water (2×10 ml) and evaporated. The solid residue is chromatographed on silica gel (hexane-ethyl acetate 3:1) to afford 2.65 g of the title compound as a red oil. MS: for C₁₅H₂₇N₂O₃ (283.4) found M⁺=283.

### Example 37

### Cmpd 37

### 2,2,5,5-Tetramethyl-3-oxiranylmethyl-imidazolidin-4-one-N-oxyl

2,2,5,5-Tetramethyl-imidazolidin-4-one-1-N-oxyl (Cmpd 2) (7.0 g, 0.045 mol) are dissolved in THF (48 ml). sodium hydride (1.23 g, 0.051 mol) is added in portions at room temperature. The mixture is heated to 30° C. and stirred for 4 h, then the solvent is removed under reduced pressure. Epichlorohydrine (42 ml) is added and the suspension stirred at 60° C. for 18 h. The solvent is removed under reduced pressure and the residue purified by flash chromatography on SiO₂ to afford 7.19 g of an orange solid, mp. 64-75° C.

### Example 38

### Cmpd 38

prepared as described in: Vanifatova, N. G.; Evstiferov, M. V.; Martin, V. V.; Petrukhin, O. M.; Volodarskii, L. B.; Zolotov, Yu. A. Zhurnal Analiticheskoi Khimii (1988), 43(3), 435-40.

### Example 39

### Cmpd 39

### 3,3,5,5-Tetramethyl-thiomorpholine 1,1-dioxide-N-oxyl

This compound is prepared as described in DE 2 351 865, p. 49, Example 6.

### Example 40

### Cmpd 40

### 2,2,7,7-Tetramethyl-1,4-diazacycloheptan-5-one 1-N-oxyl

This compound is prepared as described by Rozantsev, E. G.; Chudinov, A. V.; Sholle, V. D.: lzvestiya Akademii Nauk SSSR, Seriya Khimicheskaya (1980), (9), 2114-17.

### Example 41

### Cmpd 41

### 2,2,4,7,7-Pentamethyl-1,4-diazacycloheptan-5-one 1-N-oxyl

The solution of 2,2,7,7-tetramethyl-1,4-diazacycloheptan-5-one 1-N-oxyl (1.3 g, 7 mmol) in methyl iodide (2 ml) is stirred at room temperature with aqueous sodium hydroxide (2 ml, 50% solution) and tetrabutylammonium bromide (0.1 g) for 1 h. The organic layer is separated, washed with water and evaporated. The residue is chromatographed on silica gel with CH₂Cl₂-ethyl acetate-methanol 5:4:1 to afford 0.79 g of the title compound as a red oil which solidifies slowly on standing. MS for C₁₀H₁₉N₂O₂ (199.27) found M⁺=199.

### Example 42

### Cmpd 42

### 1,1,3,3,5,5-Hexamethyl-perhydro-1,4-diazepin-1-ium iodide-4-N-oxyl

This compound is prepared as described by Ramasseul, R.; Rassat A.; Rey, P.: Tetrahedron Letters 839 (1975).

### Example 43

### Cmpd 43

### 2,7-Diethyl-2,3,7-trimethyl-1,4-diazacycloheptan-5-one 1-N-oxyl

This compound is prepared as described in U.S. Pat. No. 6,479,608 B1, Example C3

### Example 44

### Cmpd 44

### 3,5-Diethyl-2,3,5-trimethyl-7-oxo-perhydro-1,4-diazepine-1-carboxylic acid-t-butyl ester-4-N-oxyl

This compound is prepared as described in U.S. Pat. No. 6,479,608 B1, Example C8.

### Example 45

### Cmpd 45

### 2,2,6,6-Tetramethyl-4-phenyl-perhydro-1,4-azaphosphorine 4-oxide-N-oxyl

This compound is prepared as described by Skolimowski, J.; Skowronski, R.; Simalty, M.: Tetrahedron Letters 4833-4 (1974).

### Example 46

### Cmpd 46

### 2,2,6,6-Tetramethyl-4,4-diphenyl-1,4-azatetrahydrophosphorinium perchlorate-N-oxyl

This compound is prepared as described by Skolimowski, J.; Skowronski, R.; Simalty, M.: Tetrahedron Letters 4833-4 (1974).

### Redox Potential Data for Several Compounds:

| Cmpd No. | E₀ (V) |
|---|---|
| TEMPO | 0.695 |
| 1 | 1.181 |
| 9 | 1.142 |
| 15 | 0.901 |
| 16 | 0.871 |
| 20 | 0.969 |
| 25 | 1.099 |
| 26 | 1.188 |
| 29 | 1.265 |
| 30 | 1.255 |
| 31 | 1.273 |
| 32 | 1.032 |
| 34 | 0.931 |
| 35 | 1.231 |
| 36 | 1.061 |
| 37 | 1.186 |
| 39 | 1.039 |
| 40 | 0.791 |
| 41 | 0.789 |
| 43 | 0.783 |

It can be clearly seen that compounds according to present invention have significantly higher oxidation potential than the state-of-the art compound TEMPO (2,2,6,6-tetramethylpiperidin-N-oxyl).

### Experimental Details for Redox Potential Measurement and Recording of Cyclovoltammograms

Cyclic voltammetry (CV) is performed using a three-electrode glass cell with working electrode, counter electrode and reference electrode and a computer-controlled potentiostat, applying a linear potential sweep (see e.g. B. Schoellhorn et al., New Journal of Chemistry, 2006, 30, 430-434; CAN 144:441363). Multiple CV-scans per compound used are recorded and the mean value for the peak potential is taken.

### CV—Experimental Conditions

Potentiostat: VersaStat II (EG&G Instruments), 0.1M Bu₄NBF₄, 2.7E-3M nitroxide, MeCN—Pt disk d=5 mm (WE), Pt wire (CE), Ag/AgCl/NaCl saturated (RE; +0.194V vs. NHE) 15−0−1.2V (TEMPO), 0−2.0V, 0.1V/s, 25° C.

The redox potential E₀ is calculated according E₀=0.5 (Eₚₐ+E_{pc})

(Eₚₐ=anodic peak potential, E_{pc}=cathodic peak potential)

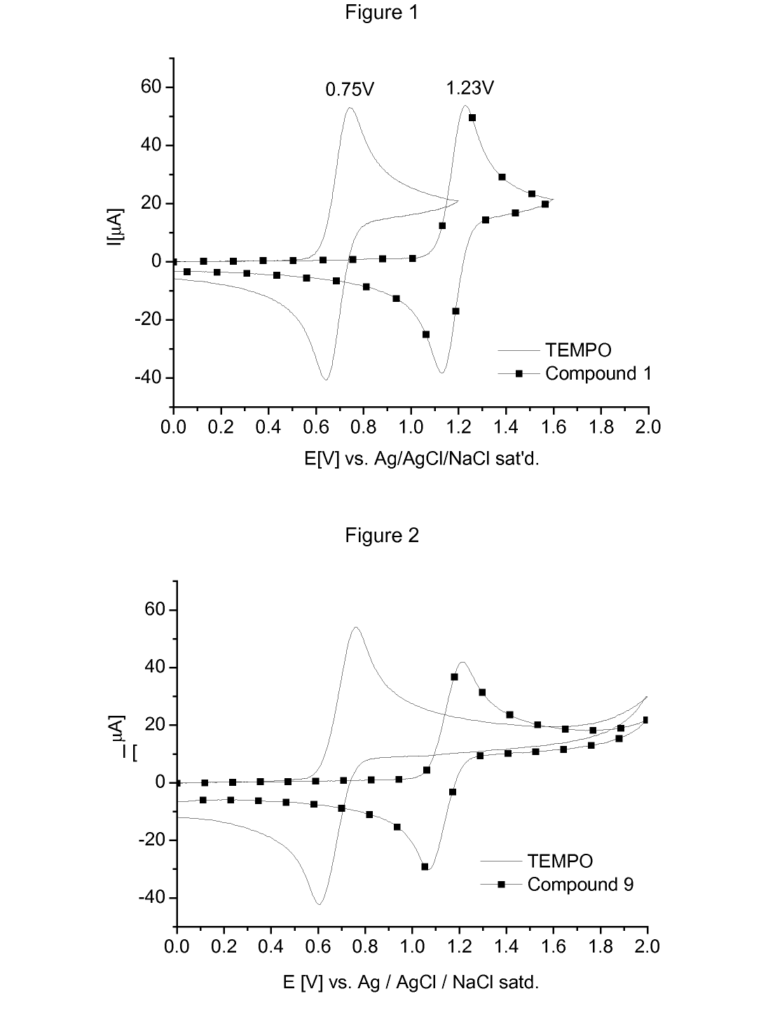

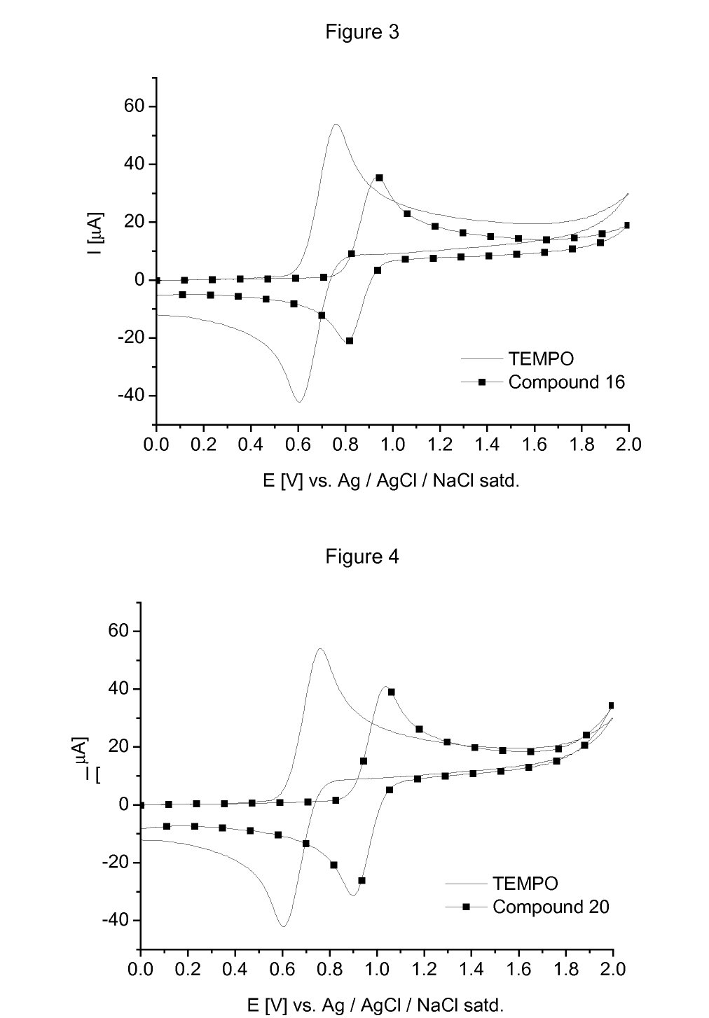

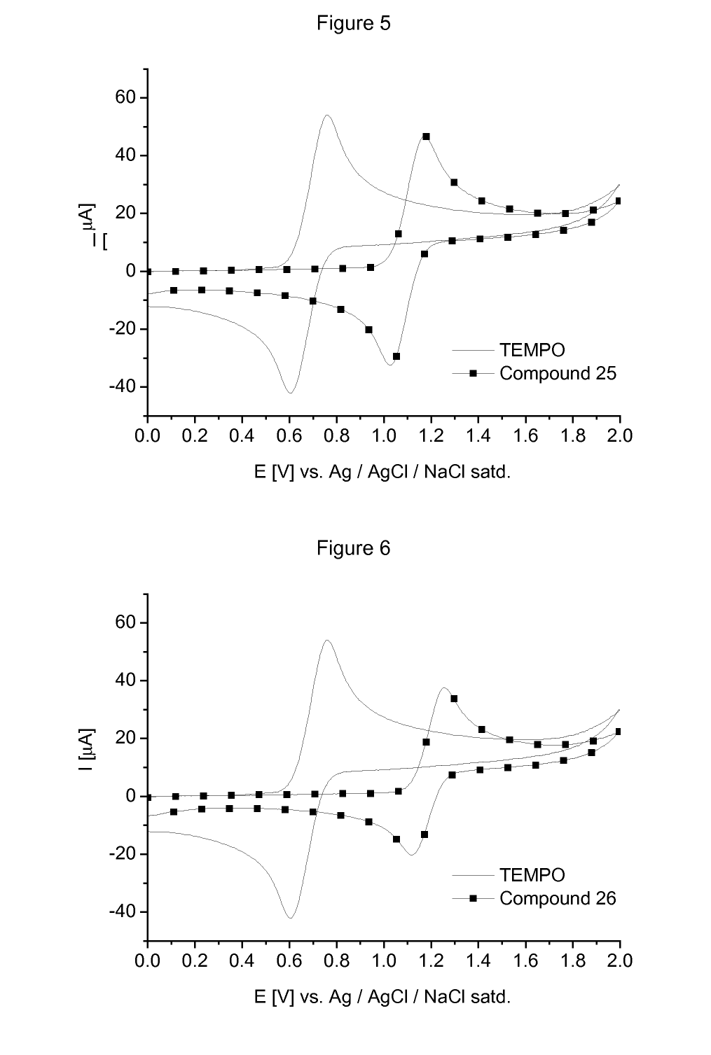

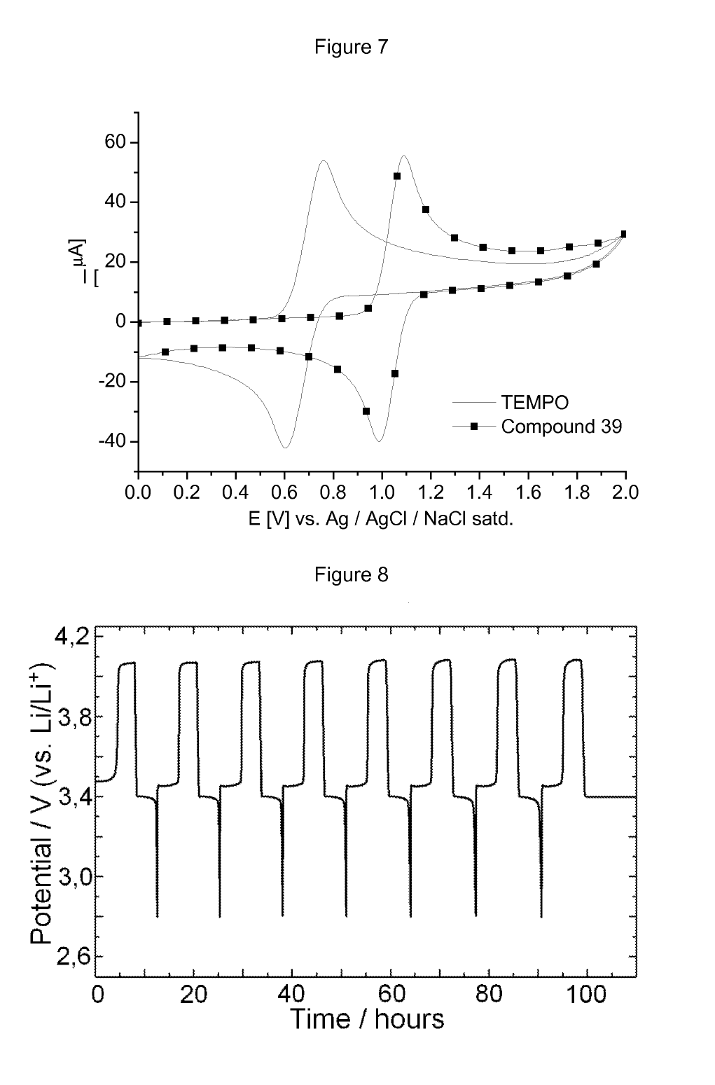

The fully reversible character of the redox process as outlined above is demonstrated by the reversible cyclovoltammograms depicted in FIGS. 1-7.

Experimental Details for Charge-Discharge and Cycling Experiments with Compound 31

Charge-discharge tests are performed using a three-electrode cell with LiFePO₄ working electrode, Li counter electrode and Li reference electrode (see e.g. J. K. Feng et al., Electrochemistry Communications, 2007, 9, 25-30; CAN 147:146607).

The positive LiFePO₄ electrode consists of 60% LiFePO₄ (Phostech), 20% Super P (Timcal), and 20% PVDF binder and is prepared by coating onto an Al foil. Li foil is used as negative electrode. The electrolyte is 1M LiPF₆ in EC/DMC 1:1 containing 0.1M compound 31. For galvanostatic experiments, the cell is repeatedly charged under constant current to 160% of the nominal charge capacity, and then discharged to 2.80 V.

The efficient overcharge protection of the Li/LiFePO₄ cell with compound 31 as electrolyte additive is demonstrated by the charge-discharge curve depicted in FIG. 8. After full charging of LiFePO₄ at 3.4 V, the voltage quickly rises above 4 V, where overcharge protection by the redox-shuttle mechanism of compound 31 sets in, resulting in a stable charge plateau at 4.1 V. This effect is maintained through 10 repeated charge-discharge cycles without any deterioration.

1. A rechargeable lithium-ion cell comprising:
(a) a positive electrode,
(b) a negative electrode and
(c) an electrolyte comprising
(i) a lithium salt,
(ii) a polar aprotic solvent and
(iii) at least one compound selected from the group consisting of formula (d1)-(d6) 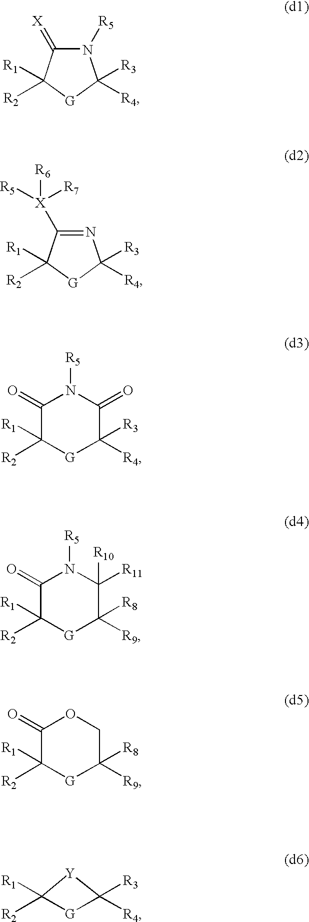
wherein
G is 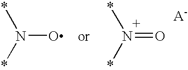
X is O or S;
when X is O,
then R₆ and R₇ are electron pairs;
when X is S,
then R₆ and R₇ are independently an electron pair or ═O;
Y is —CH₂—O—CH₂—, —CH₂—S—CH₂—, —CH₂—S(═O)—CH₂—, —CH₂—S(═O)₂—CH₂—, 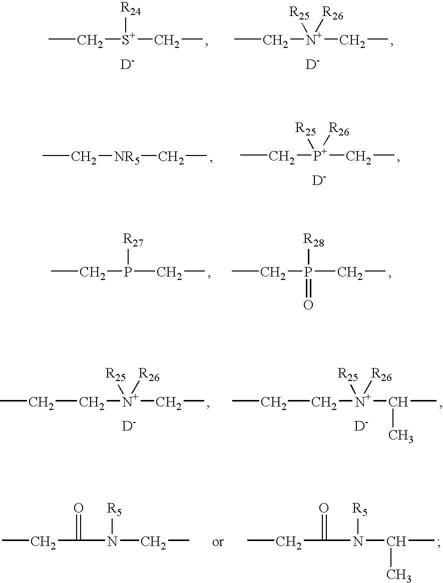
A⁻ and D⁻ are independently an anion of an organic or inorganic acid;
indicates a free valence;
R₁, R₂, R₃ and R₄ are independently C₁-C₁₈alkyl, C₆-C₁₀aryl, C₅-C₈heteroaryl, C₇-C₁₁aralkyl or C₅-C₆-cycloalkyl; said alkyl, aryl, heteroaryl, aralkyl or cycloalkyl substituted by one or more F; said alkyl and/or cycloalkyl interrupted by one or more heteroatomgroup; said alkyl and/or cycloalkyl substituted by one or more heteroatomgroup; said alkyl and/or cycloalkyl both interrupted by and substituted by one or more heteroatomgroups; or said aryl, heteroaryl and/or aralkyl substituted by 1 to 4 C₁-C₄alkyl; or
R₁ and R₂ and/or R₃ and R₄ form together with the linking carbon atom a C₄-C₁₃cycloalkylbiradical which is unsubstituted or substituted by F;
R₅ is H, OH, C₁-C₁₆, alkyl, C₆-C₁₀aryl, C₂-C₁₈alkenyl, C₂-C₁₈alkinyl, C₅-C₆cycloalkyl, glycidyl, —CO—R₁₆, —CO—NH—R₁₆, —CON(R₁₆)(R₁₇), —O—CO—R₁₆, CO—OR₁₂, —PO(OR₁₂)(OR₁₃), —S(═O)₂R₁₂, —SR₁₂, —S(═O)₂R₁₂, —S(═O)₂R₁₂, —S—OR₁₂, —S(═O)—OR₁₂, —SiR₁₆R₁₇R₁₈, —CN or halogen; said alkyl, aryl, aralkyl, alkenyl, alkinyl or cycloalkyl substituted by one or more F; said alkyl, alkenyl, alkinyl or cycloalkyl interrupted by one or more heteroatomgroup; said alkyl, alkenyl, alkinyl or cycloalkyl substituted by one or more heteroatomgroup; said alkyl and/or cycloalkyl both interrupted by and substituted by one or more heteroatomgroups; aryl or aralkyl substituted by 1 to 4 C₁-C₄alkyl; or R₅ is a multivalent core with more than one structural units (d1)-(d4) or (d6) attached;
R₈ and R₉ are independently —CH₂O—C₁-C₁₈alkyl, —Ch₂NH—CO—C₁-C₁₈alkyl or as defined for R₁; or R₈ and R₉ form together with the linking carbon atom a 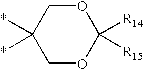
group
R₁₀ and R₁₁ are independently H or CH₃;
R₁₂ and R₁₃ are independently H, NH₄, Li, Na, K or as defined for R₁₆;
R₁₄, R₁₅ are independently H or C₁-C₈ Alkyl;
or R₁₄ and R₁₅ form together with the linking carbon atom a C₄-C₁₃cycloalkylbiradical;
R₁₆, R₁₇ and R₁₈ are independently C₁-C₁₈alkyl, C₂-C₁₈alkenyl, C₆-C₁₀aryl, C₅-C₈heteroaryl, C₇-C₁₁aralkyl or C₅-C₆cycloalkyl; said alkyl, alkenyl, aryl, heteroaryl, aralkyl or cycloalkyl substituted by one or more F; said alkyl and/or cycloalkyl interrupted by one or more heteroatomgroup; said alkyl and/or cycloalkyl substituted by one or more heteroatomgroup; said alkyl and/or cycloalkyl both interrupted by and substituted by one or more heteroatomgroups; or said aryl, heteroaryl and/or aralkyl substituted by 1 to 4 C₁-C₄alkyl;
R₂₀, R₂₁ and R₂₂ are independently C₁-C₁₈alkyl, C₂-C₁₈alkenyl, C₆-C₁₀aryl, C₅-C₈heteroaryl, C₇-C₁₁aralkyl or C₅-C₈cycloalkyl; or said alkyl, alkenyl, aryl, heteroaryl, aralkyl or cycloalkyl substituted by one or more F; and
R₂₃ is H, NH₄, Li, Na, K or as defined for R₂₀, preferably H or C₁-C₁₈alkyl;
R₂₄ is C₁-C₁₈alkyl, C₆-C₁₀aryl, C₅-C₈heteroaryl, C₇-C₁₁aralkyl, C₂-C₁₈alkenyl, C₂-C₁₈alkinyl, C₅-C₆cycloalkyl or glycidyl;
R₂₅ and R₂₆ are independently H, C₁-C₁₈alkyl, C₆-C₁₀aryl, C₇-C₁₁aralkyl, C₂-C₁₈alkenyl, C₂-C₁₈alkinyl, C₅-C₆cycloalkyl or glycidyl;
R₂₇ is C₁-C₁₈alkyl, C₆-C₁₀aryl —O—C₁-C₁₈ alkyl or —O—C₆-C₁₀aryl;
R₂₈ is H, —OH, C₁-C₁₈alkyl, C₆-C₁₀aryl, C₅-C₆cycloalkyl, —O—C₆-C₁₀aryl or —OQ, where Q is NH₄, Li, Na or K.

2. The rechargeable lithium-ion cell according to claim 1, wherein
X is O for a compound of formula (d1);
X is S for a compound of formula (d2);
Y is —CH₂—O—CH₂—, —CH₂—S—CH₂—, —CH₂—S(═O)—CH₂—, —CH₂—NR₅—CH₂—, 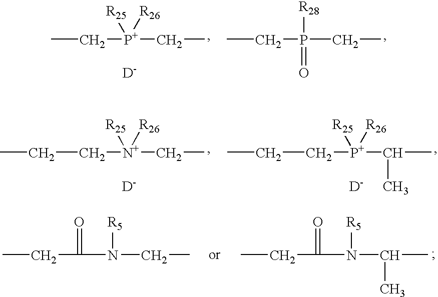
D⁻ is I⁻ or the anion of LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆H₅)₄, LiB(C₆F₅)₄, LiSbF₆, LiAsF₆, LiBr, LiBF₃C₂F₅ or LiPF₃(CF₂CF₃)₃;
R₁, R₂, R₃ and R₄ are independently C₁-C₁₈alkyl or C₆-C₁₀aryl; or said alkyl or aryl substituted by one or more F; or
R₁ and R₂ and/or R₃ and R₄ form together with the linking carbon atom a C₄-C₁₃cycloalkylbiradical which is unsubstituted or substituted by F;
R₅ is H, OH, C₁-C₁₈ alkyl, C₆-C₁₀aryl, C₃-C₁₈alkenyl, C₅-C₆cycloalkyl, glycidyl, —CO—R₁₆, —CO—NH—R₁₆, —CON(R₁₆)(R₁₇), —O—CO—R₁₆, —CO—OR₁₂, —(CH₂)_{q}COOR₁₂ or —PO(OR₁₂)(OR₁₃); said alkyl, aryl, aralkyl, alkenyl, alkinyl or cycloalkyl substituted by one or more F; or said alkyl substituted by one or more OH;
R₆ and R₇ are independently an electron pair or ═O;
R₈ and R₉ are independently —CH₂O—CO—C₁-C₁₈alkyl, —CH₂—NH—CO—C₁-C₁₈alkyl or as defined for R₁;
or R₈ and R₉ form together with the linking carbon atom a 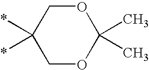
group;
R₁₀ and R₁₁ are independently H or CH₃;
R₁₂ and R₁₃ are independently H, NH₄, Li, Na, K or as defined for R₁₆,
R₁₆ and R₁₇ are independently C₁-C₁₈alkyl, C₃-C₁₈alkenyl, O₆—C₁₀aryl or C₇-C₁₁aralkyl; or said alkyl, alkenyl, aryl or aralkyl substituted by one or more F;
R₂₅ and R₂₆ are independently H, C₁-C₁₈alkyl, C₆-C₁₀aryl, C₇-C₁₁aralkyl, C₂-C₁₈alkenyl, C₂-C₁₈alkinyl, C₅-C₆cycloalkyl or glycidyl;
R₂₈ is H, —OH, C₁-C₁₈alkyl, C₆-C₁₀aryl, C₅-C₆cycloalkyl, —O—C₆-C₁₀aryl or —OQ, where Q is NH₄, Li, Na or K;
and
q is an integer from 1 to 6.

3. The rechargeable lithium-ion cell according to claim 2, wherein 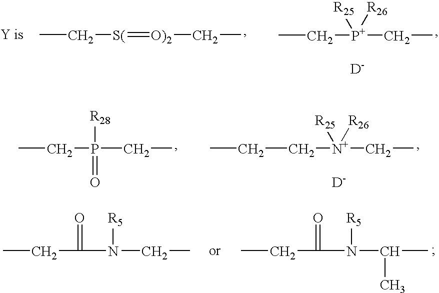
D⁻ is or ClO₄⁻;
R₁, R₂, R₃ and R₄ are independently methyl, ethyl or propyl; or
R₁ and R₂ and/or R₃ and R₄ form together with the linking carbon atom a C₆-C₇cycloalkylbiradical;
R₅ is H, OH, C₁-C₈alkyl, phenyl, benzyl, C₃-C₆alkinyl, C₅-C₆cycloalkyl, glycidyl, —CO—R₁₆, —CO—C₁-C₅ perfluoroalkyl, —CO—NH—R₁₆, CO—OR₁₆ or —PO(OR₁₂)(OR₁₃); or said alkyl substituted by one OH;
R₆ and R₇ are independently an electron pair or ═O;
R₈ and R₉ are independently —CH₂O—CO—C₁-C₄alkyl, —CH₂—NH—CO—C₁-C₄alkyl or as defined for R₁;
or R₈ and R₉ form together with the linking carbon atom a 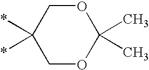
group;
R₁₀ and R₁₁ are independently H or CH₃;
R₁₂ and R₁₃ are independently H, NH₄, Li, Na, K or as defined for R₁₆; and
R₁₆ is C₁-C₈alkyl, C₃-C₆alkenyl, phenyl or benzyl;
R₂₅ and R₂₆ are C₁-C₈alkyl or phenyl; and
R₂₈ is phenyl;
with the proviso that R₅ can only be OH if R₆ and R₇ are both ═O.

4. The rechargeable lithium-ion cell according to claim 1, wherein the compound (iii) is dissolved in the electrolyte.

5. The rechargeable lithium-ion cell according to claim 1, wherein the positive electrode comprises a compound selected from the group consisting of an organic radical, LiFePO₄, Li₂FeSiO₄, Li_{w}MnO₂, MnO₂, Li₄Ti₅O₁₂, LiMnP0₄, LiCo0₂, LiNi0₂, LiNi₁₋ₓCo_{y}Met_{z}0₂, LiMn_{0.5}Ni_{0.5}0₂, LiMn_{0.3}Co_{0.3}Ni_{0.3}0₂, LiFe0₂, LiMet_{0.5}Mn_{1.5}0₄, vanadium oxide, Li₁₊ₓMn_{2-z}Met_{y}0₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof,
whereby 0<m<0.5, 0<n<0.5, 0.3≦w≦0.4, 0<x<0.3, 0<z<0.5, 0<y<0.5, Met is Al, Mg, Ti, B, Ga, Si, Ni or Co, and X is S or F.

6. The rechargeable lithium-ion cell according to claim 1, wherein the negative electrode comprises graphitic carbon, lithium metal, a lithium alloy, amorphous material based on Sn and Co, or combinations thereof.

7. The rechargeable lithium-ion cell according to claim 1, wherein the lithium salt (i) is selected from the group consisting of LiPF₆, LiClO₄, LiBF₄, LiO₃SCF₃, LiN(C₂F₅SO₂)₂, LiC(CF₃SO₂)₃, LiC(C₂F₅SO₂)₃ or LiB(C₂O₄)₂, LiB(C₆H₅)₄, LiB(C₆F₅)₄, LiSbF₆, LiAsF₆, LiBr, LiBF₃C₂F₅, LiPF₃(CF₂CF₃)₃ and combinations thereof.

8. The rechargeable lithium-ion cell according to claim 1, wherein the polar aprotic solvent (ii) is selected from the group consisting of ethylene carbonate, propylene carbonate, dimethyl carbonate, diethyl carbonate, methyl ethyl carbonate, γ-butyrolactone, tetrahydrofurane, dioxolane, sulfolane, dimethylformamide, dimethylacetamide, N-methyl-2-pyrrolidone, butylene carbonate, vinylene carbonate, fluoroethylene carbonate, fluoropropylene carbonate, methyl difluoroacetate, ethyl difluoroacetate, dimethoxyethane, bis(2-methoxyethyl)ether and combinations thereof.

9. The rechargeable lithium-ion cell according to claim 1, wherein the compound (iii) is a cyclable redox chemical shuttle which is dissolved in or is dissolvable in the electrolyte and having an oxidation potential above the recharged potential of the positive electrode.

10. The rechargeable lithium-ion cell according to claim 9, wherein the compound (iii) has an oxidation potential from 0.3 V to 5 V, above the recharged potential of the positive electrode.

11. The rechargeable lithium-ion cell according to claim 9, wherein compound (iii) provides overcharge protection after at least 30 charge-discharge cycles at a charging voltage sufficient to oxidize compound (iii), wherein G is 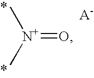
and at an overcharge charge flow equivalent to 100% of the cell capacity during each cycle.

12. The rechargeable lithium-ion cell according to claim 1, wherein the compound (iii) is a molecular redox shuttle for redox targeting.

13. The rechargeable lithium-ion cell according to claim 12, wherein the compound (iii) is dissolved in the electrolyte of the positive electrode.

14-15. (canceled)

16. A compound (d1)-(d6) according to claim 1, wherein
G is 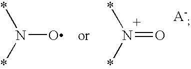
when G is 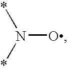
the compound is of formula (d1), (d3) or (d4) and
R₅ is —CO—R₁₆, —CON(R₁₆)(R₁₇), CO—OR₁₆, —O—CO—R₁₆, —(CH₂)_{q}COOR₁₂, —PO(OR₁₂)(OR₁₃), —S(═O)₂OR₁₂, —SR₁₂, —S(═O)₂R₁₂, —S(═O)—OR₁₂, —SiR₁₆R₁₇R₁₈, —CN or -halogen;
q is an integer from 1 to 6;
with the proviso
that for compounds of formula (d1), R₅ is —PO(OR₁₂)(OR₁₃), —S(═O)₂OR₁₂, —SR₁₂, —S(═O)R₁₂, —S(═O)₂R₁₂, —S(═O)—OR₁₂ or —SiR₁₆R₁₇R₁₈; and
that the compounds 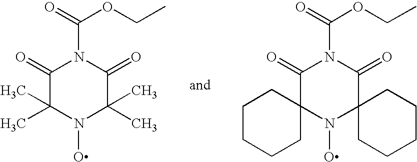
are excluded.

17. The rechargeable lithium-ion cell according to claim 1, wherein G is

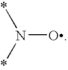

18. The rechargeable lithium-ion cell according to claim 2, wherein the compound (iii) is dissolved in the electrolyte.

19. The rechargeable lithium-ion cell according to claim 5, wherein the negative electrode comprises graphitic carbon, lithium metal, a lithium alloy, amorphous material based on Sn and Co, or combinations thereof.
